SEQUENCE LISTING (34 sequences)

SEQ ID NO: 1 - protein, 94 aa, Homo sapiens
  UNSURE 1..94: note = 10th FN Domain of Human Fibronectin
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - skipped

SEQ ID NO: 3 - protein, 7 aa, synthetic construct
  UNSURE 1..7: note = CD Loop Sequence of Human ICAM-2 Monobody Mb_ICAM2_S36
TGHGSAY

SEQ ID NO: 4 - protein, 8 aa, synthetic construct
  UNSURE 1..8: note = CD Loop Sequence of Human ICAM-2 Monobody Mb_ICAM2_S38
TGPASYGA

SEQ ID NO: 5 - protein, 6 aa, synthetic construct
  UNSURE 1..6: note = Consensus FG Loop Sequence of Human ICAM-2 Monobodies
  UNSURE 1: note = X at position 1 is Y or K
  UNSURE 3: note = X at position 3 is K or R
XWXYSP

SEQ ID NO: 6 - protein, 8 aa, synthetic construct
  UNSURE 1..8: note = FG Loop Sequence of Human ICAM-2 Monobody Mb_ICAM2_S32
NQYWKYSP

SEQ ID NO: 7 - protein, 9 aa, synthetic construct
  UNSURE 1..9: note = FG Loop Sequence of Human ICAM-2 Monobody Mb_ICAM2_S36
LWYKGITSP

SEQ ID NO: 8 - protein, 9 aa, synthetic construct
  UNSURE 1..9: note = FG Loop Sequence of Human ICAM-2 Monobody Mb_ICAM2_S38
ISNKWKYSP

SEQ ID NO: 9 - protein, 9 aa, synthetic construct
  UNSURE 1..9: note = FG Loop Sequence of Human ICAM-2 Monobody Mb_ICAM2_S40
LSSKWRYSP

SEQ ID NO: 10 - protein, 90 aa, synthetic construct
VSSVPTKLEVVAATPTSLLISWDAPAVTVLYYFITYGETGGNSPVQEFAVPGSKSTATISGLKPGVDYTITVYAINQYWKYSPISINYRT

SEQ ID NO: 11 - protein, 90 aa, synthetic construct
  UNSURE 1..90: note = Mb_ICAM2_S36
VSSVPTKLEVVAATPTSLLISWDAPAVTVLYYFITYGETGHGSAYQEFAVPGSKSTATISGLKPGVDYTITVYALWYKGITSPISINYRT

SEQ ID NO: 12 - protein, 91 aa, synthetic construct
  UNSURE 1..91: note = Mb_ICAM2_S38
VSSVPTKLEVVAATPTSLLISWDAPAVTVLYYFITYGETGPASYGAQEFAVPGSKSTATISGLKPGVDYTITVYAISNKWKYSPISINYRT

SEQ ID NO: 13 - protein, 90 aa, synthetic construct
  UNSURE 1..90: note = Mb_ICAM2_S40
VSSVPTKLEVVAATPTSLLISWDAPAVTVLYYFITYGETGGNSPVQEFAVPGSKSTATISGLKPGVDYTITVYALSSKWRYSPISINYRT

SEQ ID NO: 14 - protein, 7 aa, synthetic construct
  UNSURE 1..7: note = Shared BC Loop Sequence for Human ICAM-2 Monobodies
APAVTVL

SEQ ID NO: 15 - protein, 7 aa, synthetic construct
  UNSURE 1..7: note = Shared CD Loop Sequence for Human ICAM-2 Monobodies Mb_ICAM2_S32 and Mb_ICAM2_S40
TGGNSPV

SEQ ID NO: 16 - protein, 5 aa, synthetic construct
  UNSURE 1..5: note = Shared DE Loop Sequence for Human ICAM-2 Monobodies
PGSKS

SEQ ID NO: 17 - DNA, 273 bp, synthetic construct
  source 1..273: mol_type = other DNA
  unsure 1..273: note = DNA encoding Mb_ICAM2_S32
gtgtccagcgtgcccaccaagctggaagtggtcgccgctacacctaccagcctgctgatcagctgggatgcccctgctgttacagtgctgtactacttcatcacctacggcgagacaggcggcaacagccctgtgcaggagttcgccgtgccaggatctaagtctacagccaccatctccggcctgaaacctggcgtggactacaccattaccgtgtacgccatcaaccagtactggaagtacagccccatcagcatcaattatagaacctaa

SEQ ID NO: 18 - DNA, 273 bp, synthetic construct
  source 1..273: mol_type = other DNA
  unsure 1..273: note = DNA encoding Mb_ICAM2_S36
gttagctctgtgcctaccaagctggaagtggtggctgctacacctaccagcctgctgatctcctgggatgccccagccgtgacagtgctgtactacttcatcacctacggcgagacaggccacggcagcgcctaccaggagttcgccgtgcccggcagcaaaagcaccgccaccatttccggactgaagcctggcgtcgactacacaatcaccgtgtacgccctgtggtacaagggcatcaccagccccatctctatcaactatagaacctaa

SEQ ID NO: 19 - DNA, 276 bp, synthetic construct
  source 1..276: mol_type = other DNA
  unsure 1..276: note = DNA encoding Mb_ICAM2_S38
gtttctagcgtgcccaccaagctggaagtggtggccgctacacctaccagcctgctgatcagctgggatgcccctgctgtgaccgtgctgtactacttcatcacatacggcgagacaggccctgccagctacggcgcccaggagttcgccgtgccaggcagcaagtccaccgccacaatttctggcctgaaacctggagtggactacaccatcaccgtctacgccatctccaacaagtggaagtacagccccatcagcatcaactatagaacctaa

SEQ ID NO: 20 - DNA, 273 bp, synthetic construct
  source 1..273: mol_type = other DNA
  unsure 1..273: note = DNA encoding Mb_ICAM2_S40
gtgtccagcgtgcccaccaagctggaagtggtcgccgctacccctaccagcctgctgatcagctgggatgcccctgctgttacagtgctgtactacttcatcacctacggcgagacaggcggcaacagccctgtgcaggagttcgccgtgccaggcagcaagtctacagccacaatcagcggactgaagcctggcgtggactacaccattaccgtgtacgccctgagctctaaatggcggtacagccccatctccatcaactatagaacctaa

SEQ ID NO: 21 - protein, 92 aa, synthetic construct
  UNSURE 1..92: note = Mb_pICAM2_L1
VSSVPTKLEVVAATPTSLLISWDAGYWSSVAYYRITYGETGGNSPVQEFTVPGSSSTATISGLKPGVDYTITVYAMTSGYSWYSPISINYRT

SEQ ID NO: 22 - protein, 92 aa, synthetic construct
  UNSURE 1..92: note = Mb_pICAM2_L2
VSSVPTKLEVVAATPTSLLISWDAEYWVSVMYYRITYGETGGNSPVQEFTVPYSSYTATISGLKPGVDYTITVYAQTSMYSWYSPISINYRT

SEQ ID NO: 23 - protein, 92 aa, synthetic construct
  UNSURE 1..92: note = Mb_pICAM2_L3
VSSVPTKLEVVAATPTSLLISWDAGYWSSVAYYRITYGETGGNSPVQEFTVPSSSSTATISGLKPGVDYTITVYATTSQYSWYSPISINYRT

SEQ ID NO: 24 - protein, 92 aa, synthetic construct
  UNSURE 1..92: note = Mb_pICAM2_L5
VSSVPTKLEVVAATPTSLLISWDAGYWSSVAYYRITYGETGGNSPVQEFTVPYYSYTATISGLKPGVDYTITVYATTSQYSWYSPISINYRT

SEQ ID NO: 25 - protein, 92 aa, synthetic construct
  UNSURE 1..92: note = Mb_pICAM2_L6
VSSVPTKLEVVAATPTSLLISWDAGYWSSVAYYRITYGETGGNSPVQEFTVPSSSSTATISGLKPGVDYTITVYAQTSMYSWYSPISINYRT

SEQ ID NO: 26 - protein, 92 aa, synthetic construct
  UNSURE 1..92: note = Mb_pICAM2_L7
VSSVPTKLEVVAATPTSLLISWDAEYYSSVHYYRITYGETGGNSPVQEFTVPGSSSTATISGLKPGVDYTITVYAMTSGYSWYSPISINYRT

SEQ ID NO: 27 - protein, 92 aa, synthetic construct
  UNSURE 1..92: note = Mb_pICAM2_L10
VSSVPTKLEVVAATPTSLLISWDAEYWVSVMYYRITYGETGGNSPVQEFTVPYSSYTATISGLKPGVDYTITVYATTSQYSWYSPISINYRT

SEQ ID NO: 28 - protein, 92 aa, synthetic construct
  UNSURE 1..92: note = Mb_pICAM2_L11
VSSVPTKLEVVAATPTSLLISWDAGYWSSVAYYRITYGETGGNSPVQEFTVPSSSSTATISGLKPGVDYTITVYAMTSGYSWYSPISINYRT

SEQ ID NO: 29 - protein, 92 aa, synthetic construct
  UNSURE 1..92: note = Mb_pICAM2_L13
VSSVPTKLEVVAATPTSLLISWDAGYWSSVAYYRITYGETGGNSPVQEFTVPSSSSTATISGLKPGVDYTITVYAQTSYYSWYSPISINYRT

SEQ ID NO: 30 - protein, 92 aa, synthetic construct
  UNSURE 1..92: note = Mb_pICAM2_L14
VSSVPTKLEVVAATPTSLLISWDAGYWSSVSYYRITYGETGGNSPVQEFTVPGSSYTATISGLKPGVDYTITVYAQTSYYSWYSPISINYRT

SEQ ID NO: 31 - protein, 92 aa, synthetic construct
  UNSURE 1..92: note = Mb_pICAM2_L22
VSSVPTKLEVVAATPTSLLISWDAGYWSSVAYYRITYGETGGNSPVQEFTVPGSSYTATISGLKPGVDYTITVYAKTSDYSWYSPISINYRT

SEQ ID NO: 32 - protein, 92 aa, synthetic construct
  UNSURE 1..92: note = Mb_pICAM2_L27
VSSVPTKLEVVAATPTSLLISWDAGYWSSVAYYRITYGETGGNSPVQEFTVPGSYSTATISGLKPGVDYTITVYAQTSMYSWYSPISINYRT

SEQ ID NO: 33 - protein, 92 aa, synthetic construct
  UNSURE 1..92: note = Mb_pICAM2_L32
VSSVPTKLEVVAATPTSLLISWDAEEWSSVSYYRITYGETGGNSPVQEFTVPYYSSTATISGLKPGVDYTITVYATTSQYSWYSPISINYRT

SEQ ID NO: 34 - protein, 94 aa, synthetic construct
  UNSURE 1..94: note = Negative control monobody
VSSVPTKLEVVAATPTSLLISWDASSSSVSYYRITYGETGGNSPVQEFTVPGSKSTATISGLKPGVDYTITVYASSSSSSSSSSSKPISINYRT

(19) United States
(12) Patent Application Publication
(10) Pub. No.: US 2026/0250391 A1
(43) Pub. Date: Aug. 27, 2026

(54) MONOBODIES BINDING TO INTERCELLULAR ADHESION MOLECULE 2 (ICAM-2)

(71) Applicant: NEW YORK UNIVERSITY, New York, NY (US)

(72) Inventors: Shohei KOIDE, New York, NY (US); Akiko KOIDE, New York, NY (US)

(21) Appl. No.: 18/845,545

(22) PCT Filed: Mar. 24, 2023
(86) PCT No.: PCT/US2023/064948

§ 371 Date: Sep. 10, 2024

Related U.S. Application Data
(60) Provisional application No. 63/323,525, filed on Mar. 25, 2022.

(51) Int. Cl. C07K 16/28; A61K 31/56; A61K 31/7105; A61K 38/20; A61K 38/21; A61K 41/00; A61K 45/06; A61K 47/68; A61K 49/00; C07K 14/78; C12N 15/63

(52) U.S. Cl. CPC C07K 16/2821; A61K 31/56; A61K 31/7105; A61K 38/20; A61K 38/21; A61K 41/00; A61K 45/06; A61K 47/6811; A61K 49/00; C07K 14/78; C12N 15/63; A61K 2123/00; C07K 2317/92; C07K 2319/00

(57) ABSTRACT

The present application relates to an Intercellular Adhesion Molecule 2 (ICAM-2) binding polypeptide. This ICAM-2 binding polypeptide comprises a fibronectin type III (FN3) domain having at least one modified loop amino acid sequence and, optionally, a modified beta strand. The one or more modified loop sequences, together with the optional beta strand modifications, enable selective binding to ICAM-2. Also disclosed are conjugates that include the ICAM-2 binding polypeptide, polynucleotides encoding the same, and methods of using these materials for inhibiting transplant organ rejection as well as treatment of hypertension and cancer.

This application claims the priority benefit of U.S. Provisional Patent Application Ser. No. 63/323,525, filed Mar. 25, 2022, which is hereby incorporated by reference in its entirety.

### SEQUENCE LISTING

The Sequence Listing is being submitted electronically in XML format and is hereby incorporated by reference in its entirety. Said XML copy, created on Mar. 16, 2023, is named 147462002351.xml and is 44.3 KB in Size. No new matter is being introduced

### FIELD

The present application is directed to polypeptides that exhibit the capacity to bind specifically to Intercellular Adhesion Molecule 2 (ICAM-2), conjugates and compositions that include those polypeptides, as well as various uses thereof.

### BACKGROUND

Intercellular Adhesion Molecule 2 (ICAM-2) is constitutively expressed on vascular endothelial cells that form the lining of blood vessels (Cowan et al., “The Human ICAM-2 Promoter Is Endothelial Cell-specific in vitro and in vivo and Contains Critical Spl and GATA Binding Sites,” J. Biol. Chem. 273 (19): 11737-44 (1998)). As such, endothelial cells are the first contact point of an organ with the immune systems. Targeted delivery of therapeutics to vascular endothelial cells may be particularly effective in suppressing immune-mediated injuries to a donor organ without causing systemic toxicity to the recipient patient (Tietjen et al., “Nanoparticle Targeting to the Endothelium During Normothermic Machine Perfusion of Human Kidneys,” Sci Transl Med 9 (418): e aam6764 (2017)). One can envision that targeting to ICAM-2 may be a desirable approach to enhance delivery of therapeutics to endothelial cells. ICAM-2 is a counter receptor for lymphocyte function-associated Ag-1 (LFA-1; alphaLbeta2 integrin). ICAM-2 provides a costimulatory signal for T cell stimulation by allogenic class II MHC (Carpenito et al., “ICAM-2 Provides a Costimulatory Signal for T Cell Stimulation by Allogeneic Class II MHC,” Scand J. Immunol. 45 (3): 248-54 (1997)). Blocking the interactions between lymphocyte function associated (LFA)-1 and intercellular adhesion molecule (ICAM)-1 and ICAM-2 completely suppresses Fas-dependent B cell lysis (Wang et al., “Essential Lymphocyte Function Associated 1 (LFA-1): Intercellular Adhesion Molecule Interactions for T cell-mediated B Cell Apoptosis by Fas/APO-1/CD95,” J. Exp. Med. 186 (7): 1171-6 (1997)). Similarly, blocking ICAM-2 reduces interaction between epithelium and T cells (Porter et al., “Epithelial ICAM-1 and ICAM-2 Regulate the Egression of Human T Cells Across the Bronchial Epithelium,” FASEB J. 23 (2): 492-502 (2009)). These data suggest potential utilities of a protein that binds to ICAM-2 in drug delivery, modulation of immune cells, staining of tissues, and other applications.

It would be desirable to identify molecules capable of binding to the extracellular region of ICAM-2, particularly human ICAM-2, with high affinity and specificity. The present invention is directed to overcoming these and other deficiencies in the art.

### SUMMARY

A first aspect of the present application relates to an Intercellular Adhesion Molecule 2 (ICAM-2) binding polypeptide. This ICAM-2 binding polypeptide includes a fibronectin type III (FN3) domain having at least one modified loop amino acid sequence and, optionally, a modified beta strand. The one or more modified loop sequences, together with the optional beta strand modification, enable selective binding to ICAM-2.

A second aspect of the present application relates to an ICAM-2 binding peptide conjugate that including a first portion and a second portion. The first portion of the ICAM-2 binding peptide conjugate includes the ICAM-2 binding polypeptide according to the first aspect. The second portion of the ICAM-2 binding peptide conjugate, which is coupled to the first portion of the conjugate, is selected from a pharmaceutically active moiety, a diagnostic moiety, a half-life extending moiety, a delivery vehicle, a prodrug, a second binding molecule, a polymer, a nanoparticle, and a non-binding protein.

A third aspect of the present application relates to an isolated polynucleotide encoding the ICAM-2 binding polypeptide according to the first aspect or the disclosed ICAM-2 binding peptide conjugate according to the second aspect. Also encompassed by the third aspect are expression vectors and host cells that include the polynucleotide.

A fourth aspect of the present application relates to a pharmaceutical composition including the disclosed ICAM-2 binding polypeptide, the ICAM-2 binding peptide conjugate, or the isolated polynucleotide or vector; and a pharmaceutical carrier.

A fifth aspect of the present application relates to a combination therapeutic including the ICAM-2 binding polypeptide according to the first aspect and a pharmaceutically active agent.

A sixth aspect of the present application relates to a method of inhibiting transplant organ rejection. This method includes the step of administering to a recipient of a donor organ or tissue an effective amount of the ICAM-2 binding peptide conjugate according to the second aspect, a pharmaceutical composition according to the fourth aspect, or a combination therapeutic according to the fifth aspect, wherein the pharmaceutically active moiety or the pharmaceutically active agent is an immunosuppressant agent, whereby said administering is effective to suppress rejection of the donor organ or tissue.

A seventh aspect of the present application relates to a method of treating hypertension. This method includes the step of administering to an individual having hypertension an effective amount of the ICAM-2 binding peptide conjugate according to the second aspect, a pharmaceutical composition according to the fourth aspect, or a combination therapeutic according to the fifth aspect, wherein the pharmaceutically active moiety or the pharmaceutically active agent is an anti-hypertensive agent, and the administering is effective to treat hypertension.

An eighth aspect of the present application relates to a method of treating cancer. This method includes the step of administering to a cancer patient an effective amount of the ICAM-2 binding peptide conjugate according to the second aspect, a pharmaceutical composition according to the fourth aspect, or a combination therapeutic according to the fifth aspect, wherein the pharmaceutically active moiety or the pharmaceutically active agent is an immunostimulant, anti-angiogenic agent, or chemotherapeutic agent, and the administering is effective to treat the cancer.

A ninth aspect of the present application also relates to a method of treating cancer. This method includes the steps of administering to a cancer patient an effective amount of the ICAM-2 binding peptide conjugate according to the second aspect, wherein the conjugate includes a thermo-ablative agent; and exposing the cancer patient to energy suitable to cause localized heating of the thermo-ablative agent at the site of primary and/or secondary tumors to treat the cancer.

The present application describes the development of polypeptide monobodies that bind selectively to human ICAM-2. Using purified human ICAM-2 extracellular region as an antigen, a selection of monobody libraries was performed and four monobody clones that bound to ICAM-2 with K_{D} values in nanomolar range were identified. Biolayer interferometry measurements showed that these monobodies bound to ICAM-2 with dissociation constant (K_{D}) values ranging from 4.5 to 26 nM. These monobodies can be further modified to facilitate a particular application, such as introducing a single Cys residue for site-specific chemical reaction for immobilization to nanomaterials and for conjugating a chemical moiety (e.g., fluorescent dye or protein, active proteins such as antibodies or enzymes, or drug compounds). They can also be fused with another protein such as fluorescent proteins, antibodies, and enzymes.

### BRIEF DESCRIPTION OF THE DRAWINGS

FIG. 1 is a panel of BLI sensorgrams. The global fitting of the 1:1 binding model was performed on the data after excluding the highest concentration sensorgrams, because the association rates in the highest concentration data are too high for accurate fitting. The K_{D} values from the global fitting are shown.

FIGS. 2A-D show binding titration of Mb_ICAM2_S40 (SEQ ID NO: 13) conjugated to streptavidin DyLight 650 (SAV650) to HUVEC and Expi293 cells, as detected using flow cytometry where median fluorescence intensity is plotted (FIG. 2A), binding titration of a negative control monobody (SEQ ID NO: 34) conjugated to streptavidin DyLight 650 to HUVEC and Expi293 cells (FIG. 2B), binding titration of streptavidin DyLight 650 without a conjugated monobody to HUVEC and Expi293 cells (FIG. 2C), and detection of cell surface ICAM-2 using an anti-hICAM-2 antibody (FIG. 2D).

FIG. 3 is a Clustal Omega (version 1.2.4) alignment of four monobodies selected against human ICAM-2. The monobodies are Mb_ICAM2_S32 (SEQ ID NO: 10), Mb_ICAM2_S36 (SEQ ID NO: 11), Mb_ICAM2_S38 (SEQ ID NO: 12), and Mb_ICAM2 S40 (SEQ ID NO: 13). Variations within the CD and FG loop sequences are shown.

FIG. 4 is a Clustal Omega (version 1.2.4) alignment of thirteen monobodies selected against pig ICAM-2. The monobodies are Mb_pICAM2_L1 (SEQ ID NO: 21), Mb_pICAM2_L2 (SEQ ID NO: 22), Mb_pICAM2_L3 (SEQ ID NO: 23), Mb_pICAM2_L5 (SEQ ID NO: 24), Mb_pICAM2_L6 (SEQ ID NO: 25), Mb_pICAM2_L7 (SEQ ID NO: 26), Mb_pICAM2_L10 (SEQ ID NO: 27), Mb_pICAM2_L11 (SEQ ID NO: 28), Mb_pICAM2_L13 (SEQ ID NO: 29), Mb_pICAM2_L14 (SEQ ID NO: 30), Mb_pICAM2_L22 (SEQ ID NO: 31), Mb_pICAM2_L27 (SEQ ID NO: 32), and Mb_pICAM2_L32 (SEQ ID NO: 33). Variations within the BC, DE, and FG loop sequences are shown.

FIG. 5 is a panel of BLI sensorgrams of monobody clones binding to pig ICAM2. The deduced K_{D} values are shown.

### DETAILED DESCRIPTION

The present invention relates generally to Intercellular Adhesion Molecule 2 (ICAM-2) binding polypeptides, polynucleotides encoding the binding polypeptides, ICAM-2 binding peptide conjugates, and polynucleotides encoding the ICAM-2 binding peptide conjugates. Compositions, particularly pharmaceutical compositions containing such ICAM-2 binding polypeptides, ICAM-2 binding peptide conjugates, and polynucleotides encoding the same are also disclosed herein. The disclosure also relates to methods of using these ICAM-2 binding polypeptides, ICAM-2 binding peptide conjugates, and polynucleotides encoding the same for the delivery of diagnostic or therapeutic agents to ICAM-2-expressing tissues, including endothelial tissues.

### Definitions

Before the ICAM-2 binding polypeptides, conjugates, polynucleotides, compositions and methods are described, it is to be understood that this invention is not limited to the particular polypeptides, conjugates, polynucleotides, compositions or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred materials and methods are now described.

As used herein, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of components but not the exclusion of any other component or group of components and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” is intended to be inclusive rather than exclusive. For example, a condition A or B is satisfied by any one of the following: A is true (or present) and B is false (or not present), A is false (or not present) and B is true (or present), and both A and B are true (or present). As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited component or group of components, but that no additional component or group of components can be added to the specified method, structure, or composition.

As used herein, the term “consists essentially of,” or variations such as “consist essentially of” or “consisting essentially of,” as used throughout the specification and claims, indicate the inclusion of any recited component or group of components, and the optional inclusion of any recited component or group of components that do not materially change the basic or novel properties of the specified method, structure or composition.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., ICAM-2 binding polypeptides or polynucleotides encoding the same), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Examples of algorithms that are suitable for determining percent sequence identity and sequence similarity are the BLAST and BLAST 2.0 algorithms, which are described in Altschul et al., J. Mol. Biol. 215:403-410 (1990); and Altschul et al., Nucleic Acids Res. 25:3389-3402 (1997), each of which is hereby incorporated by reference in its entirety. Software for performing BLAST analyses is publicly available through the National Center for Biotechnology Information. In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993), which is hereby incorporated by reference in its entirety). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector,” refers to e.g. any number of nucleic acids into which a desired sequence can be inserted, e.g., be restriction and ligation, for transport between genetic environments or for expression in a host cell. Nucleic acid vectors can be DNA or RNA. Vectors include, but are not limited to, plasmids, phage, phagemids, bacterial genomes, virus genomes, self-amplifying RNA, replicons.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed polypeptide can be within the cytoplasm of a host cell, secreted into the extracellular milieu such as the growth medium of a cell culture, or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component, or another therapeutic or diagnostic reagent as disclosed herein. Also included within the definition are, for example, polypeptides containing one or more analogs of an amino acid (including, for example, unnatural amino acids, etc.), as well as other modifications known in the art.

The polypeptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel. Moreover, an “isolated nucleic acid fragment” or “isolated peptide” is a nucleic acid or protein fragment that is not naturally occurring as a fragment and/or is not typically in the functional state.

### ICAM-2 Binding Polypeptides

One aspect of the disclosure relates to an ICAM-2 binding polypeptide. This ICAM-2 binding polypeptide includes a fibronectin type III (FN3) domain having at least one modified loop amino acid sequence and, optionally, a modified beta strand. The one or more modified loop sequences, together with the optional beta strand modifications, enable selective binding to ICAM-2.

The FN3 domain is an evolutionary conserved protein domain that is about 90 amino acids in length and possesses a beta sandwich structure. The beta sandwich structure of human FN3 comprises seven beta-strands, referred to as strands A, B, C, D, E, F, G, with six connecting loops, referred to as loops AB, BC, CD, DE, EF, and FG that exhibit structural homology to immunoglobulin binding domains. Three of the six loops, i.e., loops DE, BC, and FG, correspond topologically to the complementarity determining regions of an antibody, i.e., CDR1, CDR2, and CDR3. The remaining three loops are surface exposed in a manner similar to antibody CDR3. In accordance with the present disclosure, one or more of the loop regions of each FN3 domain of the binding molecule are modified to enable specific binding to ICAM-2. The one or more modified loop region sequences is preferably a modified FG loop amino acid sequence, a modified BC loop amino acid sequence, a modified DE loop amino acid sequence, or any combination of the aforementioned modified loop sequences. In addition, the FN3 domain optionally contains one or more modifications to the beta strands, more particularly at least one of the C, D, F and G beta strands, such as any two, any three, or all four of these beta strands. In certain embodiments, the FN3 domain optionally contains one or more modifications to one or both of the C and D beta strands.

As used herein “specifically binds” or “specific binding” refers to the ability of the FN3 containing binding molecule of the disclosure to bind to a predetermined antigen, i.e., an ICAM-2 with a dissociation constant (K_{D}) of about 1×10⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Typically, the FN3 domain binds to ICAM-2 with a K_{D} that is at least ten-fold less than its K_{D} for a nonspecific antigen (for example BSA or casein), as measured by biolayer interferometry (BLI) on any suitable instrument such as an Octet instrument (Sartorius).

The modified FN3 domain of the binding molecule of the present disclosure can be a FN3 domain derived from any of the wide variety of animal, yeast, plant, and bacterial extracellular proteins containing these domains. In one embodiment, the FN3 domain is derived from a mammalian FN3 domain. Exemplary FN3 domains include, for example and without limitation, any one of the 15 different FN3 domains present in human tenascin C, or the 15 different FN3 domains present in human fibronectin (FN), for example, the 10th FN3 domain. Exemplary FN3 domains also include non-natural synthetic FN3 domains, such as those described in U.S. Pat. Publ. No. 2010/0216708 to Jacobs et al., which is hereby incorporated by reference in its entirety. Individual FN3 domains are referred to by domain number and protein name, e.g., the 10th FN3 domain of fibronectin (10FN3).

In some embodiments, the FN3 domain of the binding molecule is derived from the 10th FN domain of fibronectin (10FN3). In some embodiments, the FN3 domain of the binding molecule is derived from the human 10FN3 domain. The human 10FN3 domain has the amino acid sequence of SEQ ID NO:1 as shown below. The locations of the BC (residues 24-30), CD (residues 39-45 or 40-45), DE (residues 51-55), and FG (residues 75-86) loops are identified by bold typeface with respect to the wild-type sequence of SEQ ID NO: 1. Locations of other amino acid residues referenced in this disclosure are also identified within SEQ ID NO: 1 by their position.

(SEQ ID NO: 1)
VSDVPRDLEVVAATPTSLLISWDA₂₄PAVTVR₃₀Y₃₁YR₃₃ITYGET₃₉
G₄₀GNSPV₄₅QE₄₇FT₄₉VP₅₁GSKS₅₅TATISGLKPGVDYTITVYA₇₄
V₇₅TGRGDSPASSK₈₆PISINYRT

In accordance with the present disclosure, one or more of the loop regions or selected residues within one or more of these loop regions, optionally in combination with one or more of the beta strands, are modified to enable ICAM-2 binding specificity and affinity. Suitable modifications include amino acid residue substitutions, insertions, and/or deletions. In one aspect, amino acid residues in at least one, at least two, at least three, at least four, at least five, or all six of the loop regions are altered for ICAM-2 binding specificity and affinity. In one embodiment, one or more amino acid modifications within the loop regions at or about residues 24-30 (BC loop), residues 39-45 or 40-45 (CD loop), residues 51-55 (DE loop), and residues 75-86 (FG loop) of SEQ ID NO: 1 form the ICAM-2 binding region. In another embodiment, one or more amino acid modification within any one of these loop regions enable ICAM-2 binding.

In some embodiments, the ICAM-2 binding molecule of the present disclosure binds to human ICAM-2 and comprises a modified CD loop. In some embodiments, the modified CD loop comprises the amino acid sequence of T-G-(P/H)-(G/A)-S-(Y/A)-X-(Y/A) (SEQ ID NO: 2) or G-(P/H)-(G/A)-S-(Y/A)-X-(Y/A) (residues 2-8 of SEQ ID NO: 2) where X is optional and can be Gly (G). In some embodiments, the modified CD loop is selected from any one of the modified CD loops of SEQ ID NOs: 3 or 4 (see Table 1), or a CD loop having an amino acid sequence having at least 80% sequence identity to any one of the amino acid sequences of SEQ ID NOs: 3 or 4.

In some embodiments, the ICAM-2 binding molecule of the present disclosure binds to human ICAM-2 and comprises a modified FG loop. In some embodiments, the modified FG loop comprises the amino acid sequence of (Y/K)-W-(K/R)-Y-S-P (SEQ ID NO: 5). In some embodiments, the modified FG loop is selected from any one of the modified FG loops of SEQ ID NOs: 6-9 (see Table 1), or an FG loop having an amino acid sequence having at least 80% sequence identity to any one of the amino acid sequences of SEQ ID NOs: 6-9.

TABLE 1
Human ICAM-2 Monobody Loop Sequences

| | BC Loop | SEQ | CD Loop | SEQ | DE Loop | SEQ | FG Loop | SEQ |
|---|---|---|---|---|---|---|---|---|
| Name | Sequence | ID NO: | Sequence | ID NO: | Sequence | ID NO: | Sequence | ID NO: |
| Mb_ICAM2_S32 | APAVTVL | 14 | TGGNSPV | 15 | PGSKS | 16 | NQYWKYSP | 6 |
| Mb_ICAM2_S36 | APAVTVL | 14 | TGHGSAY | 3 | PGSKS | 16 | LWYKGITSP | 7 |
| Mb_ICAM2_S38 | APAVTVL | 14 | TGPASYGA | 4 | PGSKS | 16 | ISNKWKYSP | 8 |
| Mb_ICAM2_S40 | APAVTVL | 14 | TGGNSPV | 15 | PGSKS | 16 | LSSKWRYSP | 9 |

As discussed above, FN3 domains contain two sets of CDR-like loops on the opposite faces of the molecule. The two sets of loops are separated by beta-strands (regions of the domain that are between the loops) that form the center of the FN3 structure. Like the loops, these beta-strands can be altered to improve stability and/or enhance target molecule binding specificity and affinity. Preferably, some or all of the surface exposed residues in the beta strands are randomized without affecting (or minimally affecting) the inherent stability of the FN3 domain. In some embodiments, one or more of residues in one or more beta-strands is modified to enable interaction with ICAM-2. Suitable modifications include amino acid substitutions, insertions, and/or deletions. For example, one or more amino acid residues of the A beta strand, the B beta strand, the C beta strand, the D beta strand, the E beta strand, the F beta strand, or the G beta strand may be modified to enable ICAM-2 binding or to enhance the specificity or affinity of ICAM-2 binding. In one embodiment, one or more amino acid residues of the A, B, C, D, E, F, and/or G beta-strands are modified for binding to ICAM-2.

In some embodiments, the ICAM-2 binding polypeptide described herein comprises one or more amino acid residue substitutions, additions, or deletions in the A beta strand or region upstream thereof.

In certain embodiments, the ICAM-2 binding polypeptide described herein comprises one or more amino acid residue substitutions, additions, or deletions in the C and/or D beta strands.

In some embodiments, the ICAM-2 binding polypeptide described herein comprises one or more amino acid residue substitutions, additions, or deletions in the C beta strand thereof. In some embodiments, the ICAM-2 binding polypeptide comprises an amino acid substitution at one or more residues corresponding to residues Y31 and/or R33 of SEQ ID NO: 1. In some embodiments, the amino acid substitution is tyrosine to phenylalanine substitution at the amino acid residue corresponding to the tyrosine at position 31 (Y31F) of SEQ ID NO: 1. In some embodiments, the amino acid substitution is arginine to aspartic acid substitution at the amino acid residue corresponding to the arginine at position 33 (R33D) of SEQ ID NO: 1, arginine to valine substitution at the amino acid residue corresponding to the arginine at position 33 (R33V) of SEQ ID NO: 1, or arginine to phenylalanine substitution at the amino acid residue corresponding to the arginine at position 33 (R33F) of SEQ ID NO: 1. In some embodiments, the Y31 and/or R33 substitution is one of the substitutions listed in Table 2 in the accompanying Examples.

In some embodiments, the ICAM-2 binding polypeptide described herein comprises one or more amino acid residue substitutions, additions, or deletions in the D beta strand thereof. In some embodiments, the ICAM-2 binding polypeptide comprises an amino acid substitution at one or more residues corresponding to residues E47 and/or T49 of SEQ ID NO: 1. In some embodiments, the amino acid substitution is glutamic acid to threonine substitution at the amino acid residue corresponding to the glutamic acid at position 47 (E47T) of SEQ ID NO: 1. In some embodiments, the amino acid substitution is threonine acid to lysine substitution at the amino acid residue corresponding to the threonine at position 49 (T49K) of SEQ ID NO: 1. In some embodiments, the amino acid substitution is threonine acid to alanine substitution at the amino acid residue corresponding to the threonine at position 49 (T49A) of SEQ ID NO: 1. In some embodiments, the E47 and/or T49 substitution is one of the substitutions listed in Table 2 in the accompanying Examples.

In some embodiments, the ICAM-2 binding polypeptide described herein comprises one or more amino acid residue substitutions, additions, or deletions in the F beta strand thereof. In some embodiments, the ICAM-2 binding polypeptide comprises an amino acid substitution at one or more residues corresponding to residues A74 of SEQ ID NO: 1. In some embodiments, the amino acid substitution is alanine to threonine acid substitution at the amino acid residue corresponding to the alanine at position 74 (A74T) of SEQ ID NO: 1. In some embodiments, the A74 substitution is one of the substitutions listed in Table 2 in the accompanying Examples.

In some embodiments, the FN3 domain comprises an amino acid sequence that is at least 80% identical to an amino acid sequence of SEQ ID NO: 10. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 10. In some embodiments, the FN3 domain comprises an amino acid sequence of SEQ ID NO: 10 (Mb_ICAM2_S32) as follows:

VSSVPTKLEVVAATPTSLLISWDAPAVTVLYYFITYGETGGNSPVQEFA
VPGSKSTATISGLKPGVDYTITVYAINQYWKYSPISINYRT

In SEQ ID NO: 10, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 10 for binding to ICAM-2. For example, the A strand includes D3S, R6T, and D7K substitutions, the C strand includes a R33F substitution, and the D strand includes a T49A substitution.

In some embodiments, the FN3 domain comprises an amino acid sequence that is at least 80% identical to an amino acid sequence of SEQ ID NO: 11. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 11. In some embodiments, the FN3 domain comprises an amino acid sequence of SEQ ID NO: 11 (Mb_ICAM2_S36) as follows:

VSSVPTKLEVVAATPTSLLISWDAPAVTVLYYFITYGETGHGSAYQEFA
VPGSKSTATISGLKPGVDYTITVYALWYKGITSPISINYRT

In SEQ ID NO: 11, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 11 for binding to ICAM-2. For example, the A strand includes D3S, R6T, and D7K substitutions, the C strand includes a R33F substitution, and the D strand includes a T49A substitution.

In some embodiments, the FN3 domain comprises an amino acid sequence that is at least 80% identical to an amino acid sequence of SEQ ID NO: 12. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 12. In some embodiments, the FN3 domain comprises an amino acid sequence of SEQ ID NO: 12 (Mb_ICAM2_S38) as follows:

VSSVPTKLEVVAATPTSLLISWDAPAVTVLYYFITYGETGPASYGAQEF
AVPGSKSTATISGLKPGVDYTITVYAISNKWKYSPISINYRT

In SEQ ID NO: 12, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 12 for binding to ICAM-2. For example, the A strand includes D3S, R6T, and D7K substitutions, the C strand includes a R33F substitution, and the D strand includes a T49A substitution.

In some embodiments, the FN3 domain comprises an amino acid sequence that is at least 80% identical to an amino acid sequence of SEQ ID NO: 13. In some embodiments, the FN3 domain comprises an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 13. In some embodiments, the FN3 domain comprises an amino acid sequence of SEQ ID NO: 13 (Mb_ICAM2_S40) as follows:

VSSVPTKLEVVAATPTSLLISWDAPAVTVLYYFITYGETGGNSPVQEFA
VPGSKSTATISGLKPGVDYTITVYALSSKWRYSPISINYRT

In SEQ ID NO: 13, bold/italicized residues are modified from the wildtype 10FN3 domain of SEQ ID NO: 1. In one embodiment, one or more amino acid residues of the A, B, C, D, E, and/or F beta-strands are modified in SEQ ID NO: 13 for binding to ICAM-2. For example, the A strand includes D3S, R6T, and D7K substitutions, the C strand includes a R33F substitution, and the D strand includes a T49A substitution.

In each of the above-identified monobodies, the C and D beta strands each comprises one point mutation relative to the wildtype 10FN3 of SEQ ID NO: 1. Specifically, the C beta strand includes an R33F substitution and the D beta strand includes a T49A substitution. Further modifications of these amino acid sequences are also contemplated, including any one or more of the alternative Y31 substitutions and E47 substitutions noted above.

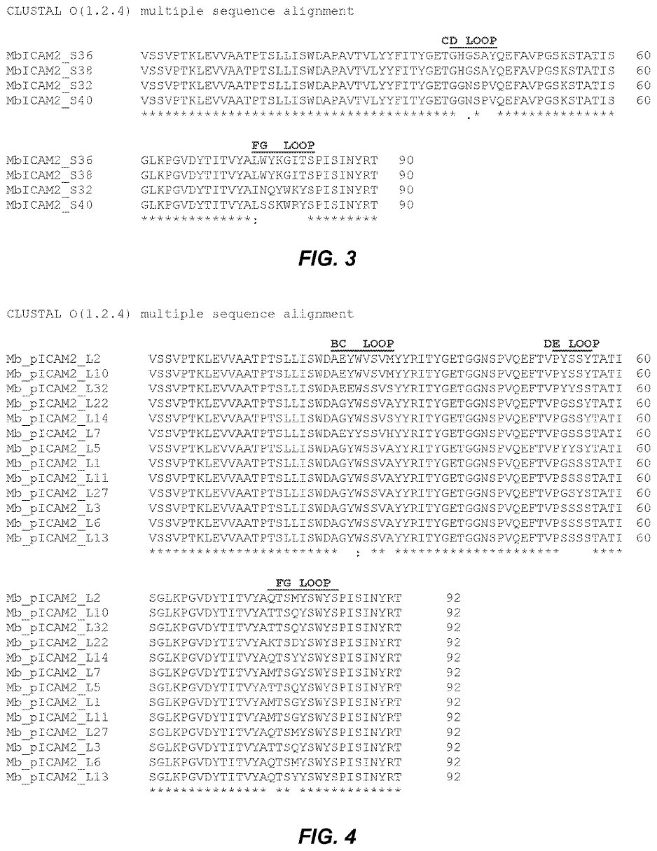

An alignment of the four human ICAM-2 binding monobodies is presented in FIG. 3.

Further modifications of these amino acid sequences are also contemplated, including any one or more of the alternative Y31 substitutions listed in Table 2, any one or more of the alternative R33 substitutions listed in Table 2, any one or more of the alternative E47 substitutions listed in Table 2, and any one or more of the alternative T49 substitutions listed in Table 2. Any one or more of these substitutions, as well as other substitutions within the C strand, D strand, F strand, or G strand as shown in Table 2, are contemplated.

According to one approach, the ICAM-2 binding polypeptide of the present invention can be synthesized by standard peptide synthesis operations. These include both FMOC (9-fluorenylmethyloxy-carbonyl) and tBoc (tert-butyloxy-carbonyl) synthesis protocols that can be carried out on automated solid phase peptide synthesis instruments including, without limitation, the Applied Biosystems 431A, 433A synthesizers, Peptide Technologies Symphony, or large scale Sonata or CEM Liberty automated solid phase peptide synthesizers. The use of alternative peptide synthesis instruments is also contemplated. Peptides prepared using solid phase synthesis are recovered in a substantially pure form.

Alternatively, as discussed below, the ICAM-2 binding polypeptide can be recombinantly produced using recombinant molecular techniques to generate host cells that contain and express a transgene that results in the production of the ICAM-2 binding polypeptide by the recombinant host cell. Upon growing the host cells under sufficient conditions to express the ICAM-2 binding polypeptide, the recombinantly produced ICAM-2 binding polypeptide can be recovered and then purified using standard techniques such as high-performance liquid chromatography, affinity chromatography, and/or size-exclusion chromatography. Other known techniques can be used alone or in combination with these chromatography techniques.

### ICAM-2 Binding Polypeptide Conjugates

Having isolated the ICAM-2 binding polypeptide, the polypeptide can used to create an ICAM-2 binding polypeptide conjugate that includes a first portion (the ICAM-2 binding polypeptide as described supra) and a second portion that is coupled to the first portion. The second portion of the ICAM-2 binding peptide conjugate, which is coupled to the first portion of the conjugate, is selected from a pharmaceutically active moiety, a diagnostic moiety, a half-life extending moiety, a delivery vehicle, a prodrug, a second binding molecule, a polymer, a nanoparticle, a non-binding protein, and any combination thereof.

In accordance with this aspect of the present disclosure, the first and second portions of the ICAM-2 binding peptide conjugate are covalently coupled to each other directly or via a linker. The first and second portions may be directly fused and generated by standard cloning and expression techniques. Alternatively, well known chemical coupling methods may be used to attach the portions directly or via a peptide or other linker to produce ICAM-2 binding peptide conjugates as described herein. For example, covalent conjugation of the first and second portions can be accomplished via lysine side chains using an activated ester or isothiocyanate, or via cysteine side chains with a maleimide, haloacetyl derivative or activated disulfide. Site specific conjugation of the first and second portions can also be accomplished by incorporating unnatural amino acids, self-labeling tags (e.g., SNAP or DHFR), or a tag that is recognized and modified specifically by another enzyme such as sortase A, lipoic acid ligase, and formylglycine-generating enzyme. In some embodiments, site specific conjugation of the first and second portions is achieved by the introduction of a cysteine residue either at the C-terminus of the ICAM-2 binding peptide (as described by Wojcik et al., “A Potent and Highly Specific FN3 Monobody Inhibitor of the Abl SH2 Domain,” Nat Struct Mol Biol. 17 (4): 519-527 (2010), which is hereby incorporated by reference in its entirety) or at a specific site (as described by Goldberg et al., “Engineering a Targeted Delivery Platform Using Centyrins,” Protein Engineering, Design & Selection 29 (12): 563-572 (2016), which is hereby incorporated by reference in its entirety).

In some embodiments, the first and second portions of the ICAM-2 binding peptide conjugate are coupled together via a linker. In some embodiments, the linker is a peptide linker. In some embodiments, the peptide linker is a cleavable linker. In some embodiments, the peptide linker is a non-cleavable linker.

Suitable linkers include peptides composed of repetitive modules of one or more of the amino acids, such as glycine and serine, or alanine and proline, or polyacidic amino acids. Exemplary linker peptides include, e.g., (Gly-Gly)n, (Gly-Ser)n, (Gly₃-Ser)n, (Ala-Pro)n wherein n is an integer from 1-25. The length of the linker may be appropriately adjusted as long as it does not affect the function of the non-binding protein-drug conjugate. The standard 15 amino acid (Gly₄-Ser)₃ linker peptide has been well-characterized and has been shown to adopt an unstructured, flexible conformation. In addition, this linker peptide does not interfere with assembly and activity of the domains it connects (Freund et al., “Characterization of the Linker Peptide of the Single-Chain Fv Fragment of an Antibody by NMR Spectroscopy,” FEBS 320:97 (1993), which is hereby incorporated by reference in its entirety). Other exemplary linkers include, e.g., (D/E)ₙ where n is an integer from 4 to 25, such as from 6 to 20 amino acids, including poly-aspartic acid and poly-glutamic acid linkers that are from 4 to 25 or 6 to 20 amino acids in length. Any of these exemplary linker peptide sequences may also be adapted with one or more cysteine residues. The linker can optionally be coupled or conjugated to a substrate, pharmaceutically active agent, or particle using, e.g., maleimide chemistry.

In one embodiment, the pharmaceutically active moiety of the ICAM-2 binding peptide conjugate is coupled to a diagnostic moiety or a pharmaceutically active moiety (i.e., a therapeutic agent).

A number of suitable diagnostic moieties can be used including, without limitation, any of a variety of small molecule fluorophores or fluorescent dyes, dendrimers, fluorescent or luminescent polypeptides (e.g., Aequorea green fluorescent protein and derivatives thereof, Anthozoan fluorescent protein and derivatives thereof, Discosoma red fluorescent protein and derivatives thereof, Anemonia fluorescent protein and derivatives thereof, firefly luciferase protein and derivatives thereof, Renilla luciferase protein and derivatives thereof, and bacterial luciferase protein and derivatives thereof), radio ligands (e.g., radionucleotide with chelator) and radioisotopes, and nanoparticle fluorophores (e.g., fluorescently doped silicas, semiconducting polymer dots, quantum dots, carbon dots, other carbonaceous nanomaterials, upconversion nanoparticles, noble metal nanoparticles (mainly gold and silver), iron oxide nanoparticles, and various other nanomaterials, contrasting agents, and photosensitizers. Such diagnostic agents may be used for in vitro or in vivo imaging, including whole or partial body imaging techniques such as Single-Photon Emission Computed Tomography, Nuclear Magnetic Resonance Imaging, Computer Assisted Tomography, Positron Emission Tomography, Positron Emission Tomography-Computed Tomography, and any variations of these techniques.

Any of a variety of pharmaceutically active moieties can be used in the conjugates of the present invention, including without limitation anti-inflammatory agents, immunomodulatory agents (e.g., immunosuppressants, immunostimulants), and anti-hypertensive agents.

Non-limiting examples of anti-inflammatory agents include steroidal and non-steroidal anti-inflammatory agents (which may also act as immunosuppressants). Exemplary non-steroidal anti-inflammatory agents include, without limitation, ibuprofen, naproxen, fenoprofen, ketoprofen, flurbiprofen, oxaprozin, indomethacin, sulindac, etodolac, diclofenac, piroxicam, meloxicam, tenoxicam, droxicam, lomoxicam, isoxicam, mefenamic acid, meclofenamic acid, flufenamic acid, tolfenamic acid, celecoxib, rofecoxib, valdecoxib, parecoxib, lumiracoxib, etoricoxib, and aspirin.

Exemplary steroids include, without limitation, betamethasone, dexamethasone, flumethasone, methylprednisolone, paramethasone, prednisolone, prednisone, triamcinolone, hydrocortisone or cortisone, alcomethasone dipropionate, amcinonide, betamethasone dipropionate, betamethasone monopropionate, betamethasone 17-valerate, budesonide, budesonide disodium phosphate, ciclomethasone, clobetasol-17-propionate, clobetasone-17-butyrate, cortisone acetate, deprodone propionate, desonide, desoxymethasone, dexamethasone acetate, diflucortolone valerate, diflurasone diacetate, diflucortolone, difluprednate, flumetasone pivalate, flunisolide, fluocinolone acetonide acetate, fluocinonide, fluocortolone, fluocortolone caproate, fluocortolone hexanoate, fluocortolone pivalate, fluormetholone acetate, fluprednidene acetate, fluticasone propionate, halcinonide, halometasone, hydrocortisone acetate, hydrocortisone-17-butyrate, hydrocortisone-17-valerate, medrysone, methylprednisolone acetate, mometasone furoate, parametasone acetate, prednicarbate, prednisolone acetate, prednylidene, rimexolone, tixocortol pivalate, triamcinolone acetonide, triamcinolone alcohol, triamcinolone hexacetonide, betamethasone sodium phosphate, desonide sodium phosphate, dexamethasone sodium phosphate, hydrocortisone sodium phosphate, hydrocortisone sodium succinate, cortisone sodium phosphate, cortisone sodium succinate, methylprednisolone disodium phosphate, methylprednisolone sodium succinate, methylprednisone disodium phosphate, methylprednisone sodium succinate, prednisolone sodium phosphate, prednisolone sodium succinate, prednisone sodium phosphate, prednisone sodium succinate, prednisolamate hydrochloride, triamcinolone acetonide disodium phosphate, or triamcinolone acetonide dipotassium phosphate.

Additional non-limiting examples of immunosuppressants include, methotrexate, sulfasalazine, D-penicillamine, nambumetone, aurothioglucose, auranofin, colloidal gold, cyclosporine, rapamycin, tacrolimus, pimecrolimus, everolimus, sirolimus, tofacitinib, azathioprine, leflunomide, mycophenolate, thalidomide, lenalidomide, etanercept, pegsunercept, bupropion, pentoxifylline, abatacept, adalimumab, anakinra, certolizumab, etanercept, golimumab, infliximab, ixekizumab, natalizumab, rituximab, secukinumab, tocilizumab, ustekinumab, vedolizumab, basiliximab, or daclizumab, as well as any active binding fragments of the above-listed antibodies.

Non-limiting examples of immunostimulants include colony stimulating factors such as filgrastim, pegfilgrastim, eflapegrastim, sargramostim, tbo-filgrastim; interferons such as interferon beta-la, peg-interferon beta-la, interferon beta-1b, interferon alfacon-1, interferon alfa-n3, interferon gamma-1b; and interleukins such as IL-2 and derivatives thereof (e.g., aldesleukin) or IL-11 and derivatives thereof (e.g., oprelvekin).

Non-limiting examples of anti-hypertensive agents include diuretics, adrenergic receptor antagonists, adrenergic receptor agonists, calcium channel blockers, Angiotensin-Converting Enzyme (ACE) inhibitors, angiotensin II receptor antagonists, aldosterone antagonists, vasodilators, and renin inhibitors.

Exemplary diuretics include, without limitation, loop diuretics such as furosemide, bumetanide, ethacrynic acid, and torsemide; thiazide diuretics such as epitizide, hydrochlorothiazide, hydroflumethiazide, chlorothiazide, bendroflumethiazide, polythiazide, trichlormethiazide, cyclopenthiazide, methyclothiazide, cyclothiazide, mebutizide, and other benzothiadiazine derivatives; thiazide-like diuretics such as indapamide, chlortalidone, metolazone, quinethazone, clopamide, mefruside, clofenamide, meticrane, xipamide, clorexolone, or fenquizone; potassium-sparing diuretics such as amiloride, triamterene, eplerenone, benzamil, potassium canrenoate, canrenone, or spironolactone; osmotic diuretics, such as mannitol, glucose, and urea; vasopressin receptor antagonists such as conivaptan, relcovaptan, nelivaptan, lixivaptan, mozavaptan, satavaptan, tolvaptan, or demeclocycline; mercurial diuretics such as mersalyl acid (Mersal), meralluride, mercaptomerin, mercurophylline, merethoxylline procaine, and calomel; xanthine diuretics such as caffeine, theobromine, paraxanthine, or theophylline; carbonic anhydrase inhibitors, such as acetazolamide, methazolamide, dorzolamide, sulfonamide, or topiramate; diuretic purines such as a diuretic steroid, a diuretic sulfonamide derivative, a diuretic uracil derivative, amanozine, arbutin, chlorazanil, etozolin, hydracarbazine, isosorbide, metochalcone, muzolimine, perhexiline, ticrynafen, triamterene, or spironolactone.

Exemplary adrenergic receptor antagonists include, without limitation, beta blockers such as atenolol, metoprolol, nadolol, oxprenolol, pindolol, propranolol, timolol, acebutolol, bisoprolol, esmolol, labetalol, carvedilol, bucindolol, nebivolol, alprenolol, amosulalol, arotinolol, befunolol, betaxolol, bevantolot, bopindolol, bucumolol, bufetolol, bufuralol, bunitrolol, bupranolol, butidrine hydrochloride, butofilolol, carazolol, carteolol, celiprolol, cetamolol, cloranololdilevalol, epanolol, indenolol, levobunolol, mepindolol, metipranolol, moprolol, nadoxolol, nipradilol, penbutolol, practolol, pronethalol, sotalol, sulfinalol, talinolol, tertatolol, tilisolol, toliprolol, and xibenolol; as well as alpha blockers such as such as phenoxybenzamine, prazosin, doxazosin, terazosin, trimazosin, phentolamine, amosulalol, arotinolol, dapiprazole, fenspiride, indoramin, labetalol, naftopidil, nicergoline, tamsulosin, tolazoline, reserpine, moxonidine or yohimbine.

Exemplary adrenergic receptor agonists include, without limitation, clonidine, methyldopa, guanfacine, methoxamine, methylnorepinephrine, oxymetazoline, phenylephrine, guanabenz, guanoxabenz, guanethidine, xylazine, and tizanidine.

Exemplary calcium channel blockers include, without limitation, dihydropyridine such as amlodipine, felodipine, nicardipine, nifedipine, nimodipine, isradipine, nitrendipine, aranidipine, barnidipine, benidipine, cilnidipine, efonidipine, elgodipine, lacidipine, lercanidipine, manidipine, nilvadipine, and nisoldipine.

Exemplary calcium channel blockers include, without limitation, non-dihydropyridines such as diltiazem, verapamil, bepridil, clentiazem, fendiline, gallopamil, mibefradil, prenylamine, semotiadil, terodiline, cinnarizine, flunarizine, lidoflazine, lomerizine, bencyclane, etafenone, and perhexiline.

Exemplary ACE inhibitors include, without limitation, sulfhydryl-containing agents such as captopril and zofenopril; dicarboxylate-containing agents such as enalapril, ramipril, quinapril, perindopril, lisinopril, and benazepril; phosphonate-containing agents such as fosinopril and ceronapril; naturally occurring ACE inhibitors such as casokinins, lactokinins; tripeptides such as Val-Pro-Pro and Ile-Pro-Pro and the nonapeptide teprotide; and additional ACE inhibitors such as alacepril, cilazapril, delapril imidapril moexipril, rentiapril, spirapril, temocapril, moveltipril or trandolapril.

Exemplary angiotensin II receptor antagonists include, without limitation, candesartan, eprosartan, irbesartan, losartan, olmesartan, telmisartan, and valsartan.

Exemplary aldosterone antagonists include, without limitation, eplerenone, canrenone, and spironolactone.

Exemplary vasodilators include, without limitation, cerebral vasodilators such as bencyclane, cinnarizine, citicoline, cyclandelate, ciclonicate, diisopropylamine dichloroacetate, eburnamonine, fasudil, fenoxedil, flunarizine, ibudilast, ifenprodil, lomerizine, nafronyl, nicametate, nicergoline, nimodipine, papaverine, tinofedrine, vincamine, vinpocetine, and viquidil; coronary vasodilators such as amotriphene, bendazol, benfurodil hemisuccinate, benziodarone, chloracizine, chromonar, clobenfural, clonitrate, cloricromen, dilazep, dipyridamole, droprenilamine, efloxate, erythrityl tetranitrate, etafenone, fendiline, floredil, ganglefene, hexestrol bis(beta-diethylaminoethyl) ether, hexobendine, itramin tosylate, khellin, lidoflazine, mannitol hexanitrate, medibazine, nitroglycerin, pentaerythritol tetranitrate, pentrinitrol, perhexiline, pimefylline, prenylamine, propatyl nitrate, trapidil, tricromyl, trimetazidine, trolnitrate phosphate, sildenafil, tadalafil, vardenafil, sodium nitroprusside, isosorbide mononitrate, isosorbide dinitrate, pentaerythritol tetranitrate, theobromine, and visnadine; peripheral vasodilators such as aluminum nicotinate, bamethan, bencyclane, betahistine, bradykinin, brovincamine, bufeniode, buflomedil, butalamine, cetiedil, ciclonicate, cinepazide, cinnarizine, cyclandelate, diisopropylamine dichloroacetate, eledoisin, fenoxedil, flunarizine, hepronicate, ifenprodil, iloprost, inositol niacinate, isoxsuprine, kallidin, kallikrein, moxisylyte, nafronyl, nicametate, nicergoline, nicofuranose, nylidrin, pentifylline, pentoxifylline, piribedil, prostaglandin E1, suloctidil, tolazoline, and xanthinol niacinate.

Exemplary renin inhibitors include, without limitation, aliskiren and remikiren.

In certain embodiments, the pharmaceutically active moiety is a cancer therapeutic agent. Non-limiting examples of cancer therapeutic agent include antimetabolites, alkaloids, alkylating agents, anti-mitotic agents, antitumor antibiotic agents, DNA binding agents, toxins, anti-proliferative agents, DNA antagonists, radionuclides, thermoablative agents, proteolysis targeting chimeras (PROTACs), and nucleic acid inhibitors.

Exemplary alkaloids include, without limitation, duocarmycin, docetaxel, etoposide, irinotecan, paclitaxel, teniposide, topotecan, vinblastine, vincristine, vindesine, and analogs and derivatives thereof.

Exemplary alkylating agents include, without limitation, busulfan, improsulfan, piposulfan, benzodepa, carboquone, meturedepa, uredepa, altretamine, triethylenemelamine, triethylenephosphoramide, triethylenethiophosphorarnide, chlorambucil, chloranaphazine, cyclophosphamide, estramustine, ifosfamide, mechlorethamine, mechlorethamine oxide HCl, melphalan, novemebichin, perfosfamide phenesterine, prednimustine, trofosfamide, uracil mustard, carmustine, chlorozotocin, fotemustine, lomustine, nimustine, semustine ranimustine, dacarbazine, mannomustine, mitobronitol, mitolactol, pipobroman, temozolomide, and analogs and derivatives thereof.

Exemplary antitumor antibiotics include, without limitation, aclacinomycin, actinomycin, anthramycin, azaserine, bleomycin, cactinomycin, calicheamicin, carubicin, carzinophilin, cromomycin, dactinomycin, daunorubicin, 6-diazo-5-oxo-1-norleucine, doxorubicin, epirabicin, idarubicin, menogaril, mitomycin, mycophenolic acid, nogalamycine, olivomycin, peplomycin, pirarubicin, plicamycin, porfiromycin, puromycine, pyrrolobenzodiazepine, streptonigrin, streptozocin, tubercidin, zinostatin, zorubicin, and analogs and derivatives thereof.

Exemplary antimetabolites include, without limitation, SN-38, denopterin, edatrexate, mercaptopurine (6-MP), methotrexate, piritrexim, pteropterin, pentostatin (2′-DCF), tomudex, trimetrexate, cladridine, fludarabine, thiamiprine, ancitabine, azacitidine, 6-azauridine, carmofur, cytarabine, doxifluridine, emitefur, floxuridine, fluorouracil, gemcitabine, tegafur, hydroxyurea, urethane, and analogs and derivatives thereof.

Exemplary anti-proliferative agents include, without limitation, aceglatone, amsacrine, bisantrene, camptothecin, defosfamide, demecolcine, diaziquone, diflomotecan, eflornithine, elliptinium acetate, etoglucid, etopside, fenretinide, gallium nitrate, hydroxyurea, lamellarin D, lonidamine, miltefosine, mitoguazone, mitoxantrone, mopidamol, nitracrine, pentostatin, phenamet, podophillinic acid 2-ethyl-hydrazide, procarbazine, razoxane, sobuzoxane, spirogermanium, teniposide, tenuazonic acid, triaziquone 2,2′,2″-trichlorotriethylamine, and analogs and derivatives thereof.

Exemplary antimitotic agents include, without limitation, an auristatin, a maytansinoid, a dolastatin, a tubulysin, a taxane, an epothilone, a vinca alkaloid, and analogs and derivatives thereof.

In some embodiments, the ICAM-2 binding peptide conjugate (that includes the ICAM-2 binding polypeptide) is coupled to a delivery vehicle that contains the pharmaceutically active moiety.

In accordance with this aspect of the disclosure, any suitable drug delivery vehicle known in the art can be coupled to the ICAM-2 binding polypeptide to form the ICAM-2 binding peptide conjugate as described herein. In any embodiment, the drug delivery vehicle is a nanoparticle delivery vehicle, a polymer-based particle, or a lipid-based particle delivery vehicle known in the art (see, e.g., Xiao et al., “Engineering Nanoparticles for Targeted Delivery of Nucleic Acid Therapeutics in Tumor,” Mol. Ther. Meth. Clin. Dev. 12:1-18 (2019) and Ni et al., “Synthetic Approaches for Nucleic Acid Delivery: Choosing the Right Carriers,” Life 9 (3): 59 (2019), which are hereby incorporated by reference in their entirety), can be employed in the methods as described herein.

Suitable nanoparticle delivery vehicles comprise, without limitation, gold nanoparticles, calcium phosphate nanoparticles, cadmium (quantum dots) nanoparticles, iron oxide nanoparticles, as well as particles derived from any other solid inorganic materials as known in the art.

Suitable polymer-based particles or polyplex carriers comprise cationic polymers such as polyethylenimine (PEI), and/or cationic polymers conjugated to neutral polymers, like polyethylene glycol (PEG) and cyclodextrin. Other suitable PEI conjugates to facilitate nucleic acid molecule or expression vector delivery in accordance with the methods described herein include, without limitation, PEI-salicylamide conjugates and PEI-steric acid conjugate. Other synthetic cationic polymers suitable for use as a delivery vehicle material include, without limitation, poly-L-lysine (PLL), polyacrylic acid (PAA), polyamideamine-epichlorohydrin (PAE) and poly [2-(dimethylamino)ethyl methacrylate] (PDMAEMA). Natural cationic polymers suitable for use as delivery vehicle material include, without limitation, chitosan, poly(lactic-co-glycolic acid) (PLGA), gelatin, dextran, cellulose, and cyclodextrin.

Suitable lipid-based vehicles include cationic lipid based lipoplexes (e.g., 1,2-dioleoyl-3trimethylammonium-propane (DOTAP)), neutral lipids based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2 (sperminecarboxamido)ethyl]-N,N-dimethyl-1-propanaminium trifluoroacetate (DOSPA)). Other suitable lipid-based delivery particles incorporate ionizable DOSPA in lipofectamine and DLin-MC3-DMA ((6Z,9Z,28Z,31Z)-heptatriaconta-6,9,28,31-tetraen-19-yl-4-(dimethylamino) butanoate).

In another embodiment, the second portion of the ICAM-2 binding peptide conjugate comprises a second polypeptide. In some embodiments, the second polypeptide is a non-binding molecule. In some embodiments, the polypeptide is a second binding molecule such as an antibody or antibody binding domain thereof.

In some embodiment, the antibody is an antibody (or antibody binding domain thereof) that binds to a tumor-specific antigen or cancer cell specific antigen. In some embodiments, the antibody is an antibody (or antibody binding domain thereof) that binds to cell surface protein expressed on oncogenic RAS cancer cells. In any embodiment, the antibody (or antibody binding domain thereof) binds to a cancer cell specific surface protein selected from CUB domain-containing protein 1 (CDCP1), Intercellular adhesion molecule 1 (ICAM1), Integrin beta-5 (ITGB5), (5′-nucleotidase) NT5E, Tumor necrosis factor receptor superfamily member 3 (LTBR), Complement decay-accelerating factor (CD55), Aminopeptidase N (ANPEP), CD79, Trophoblast glycoprotein (TPBG), Integrin beta-1 (ITGB1), Prostaglandin F2 receptor negative regulator (PTGFRN), Integrin alpha-5 (ITGA5), and Exosome complex protein LRP1 (LRP1) (see e.g., Martinko et al., “Targeting RAS-driven Human Cancer Cells with Antibodies to Upregulated and Essential Cell-Surface Proteins,” eLIFE 7: e31098 (2018), which is hereby incorporated by reference in its entirety). A number of exemplary antibodies are identified above.

As used herein, an antibody includes any protein or peptide containing molecule that comprises at least a portion of an immunoglobulin molecule, such as but not limited to, at least one, at least two, or at least three complementarity determining region (CDR) of a heavy or light chain, a heavy chain or light chain variable region, a heavy chain or light chain constant region, a framework region, or any portion thereof. The term ‘antibody’ encompass full antibodies, digestion fragments, specified portions and variants thereof, including, without limitation, portions of antibodies that mimic the structure and/or function of an antibody or specified fragment or portion thereof, including, without limitation, single chain antibodies, single domain antibodies (i.e., antibody fragments comprising merely one variable domain, which might be VHH, VH or VL, that specifically bind an antigen or epitope independently of other V regions or domains). Functional fragments of antibodies include antigen-binding fragments that bind to a particular target. For example, antibody fragments capable of binding to a particular target or portions thereof, include, but are not limited to, Fab (e.g., by papain digestion), Fab′ (e.g., by pepsin digestion and partial reduction) and F(ab′) 2 (e.g., by pepsin digestion), Fd (e.g., by pepsin digestion, partial reduction and reaggregation), Fv or scFv (e.g., by molecular biology techniques) fragments.

### Polynucleotides, Vectors, and Host Cells

Another aspect of the present disclosure is directed to polynucleotides encoding the ICAM-2 binding molecules or the ICAM-2 binding peptide conjugates described herein. The polynucleotides of the present disclosure include isolated polynucleotides, portions of expression vectors or portions of linear DNA sequences, including linear DNA sequences used for in vitro transcription/translation, vectors compatible with prokaryotic, eukaryotic or filamentous phage expression, secretion and/or display of the compositions or directed mutagens thereof, as well as linear RNA molecules (such as mRNA).

In one embodiment isolated polynucleotides of the present disclosure include those encoding the binding molecules described supra. In certain embodiments, the polynucleotides are preferably codon-optimized for expression in a particular host cell or organism as discussed below.

Exemplary isolated polynucleotide molecules include those encoding a FN3 domain comprising a modified CD loop. In some embodiments, the modified CD loop comprises the amino acid sequence of T-G-(P/H)-(G/A)-S-(Y/A)-X-(Y/A) (SEQ ID NO: 2) or G-(P/H)-(G/A)-S-(Y/A)-X-(Y/A) (residues 2-8 of SEQ ID NO: 2) where X is optional and can be Gly (G). In some embodiments, the modified CD loop is selected from any one of the modified CD loops of SEQ ID NOs: 3 or 4 (see Table 1), or a CD loop having an amino acid sequence having at least 80% sequence identity to any one of the amino acid sequences of SEQ ID NOs: 3 or 4.

Exemplary isolated polynucleotide molecules include those encoding a FN3 domain comprising a modified FG loop. In some embodiments, the modified FG loop comprises the amino acid sequence of (Y/K)-W-(K/R)-Y-S-P (SEQ ID NO: 5). In some embodiments, the modified FG loop is selected from any one of the modified FG loops of SEQ ID NOs: 6-9 (see Table 1), or an FG loop having an amino acid sequence having at least 80% sequence identity to any one of the amino acid sequences of SEQ ID NOs: 6-9.

Exemplary isolated polynucleotide molecules include those encoding a FN3 domain comprising an amino acid sequence that is at least 80% identical to the amino acid sequence of SEQ ID NO: 10. In some embodiments, the polynucleotide encodes a FN3 domain comprising an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 10. In some embodiments, the polynucleotide encodes a FN3 domain comprising the amino acid sequence of SEQ ID NO: 10 (Mb_ICAM2_S32). In these embodiments, the polynucleotide may encode one or more amino acid residues of the A, B, C, D, E, and/or F beta-strands that are modified in SEQ ID NO: 10 (from the corresponding wildtype residue) for binding to ICAM-2.

In one embodiment, the exemplary isolated polynucleotide encoding the FN3 domain of SEQ ID NO: 10 (Mb_ICAM2_S32) is a DNA molecule that is codon-optimized for expression in human cells. One such polynucleotide includes the DNA sequence according to SEQ ID NO: 17 as follows:

GTGTCCAGCGTGCCCACCAAGCTGGAAGTGGTCGCCGCTACACCTACCA
GCCTGCTGATCAGCTGGGATGCCCCTGCTGTTACAGTGCTGTACTACTT
CATCACCTACGGCGAGACAGGCGGCAACAGCCCTGTGCAGGAGTTCGCC
GTGCCAGGATCTAAGTCTACAGCCACCATCTCCGGCCTGAAACCTGGCG
TGGACTACACCATTACCGTGTACGCCATCAACCAGTACTGGAAGTACAG
CCCCATCAGCATCAATTATAGAACCTAA

This polynucleotide can be coupled to any of a variety of promoter and enhancer sequences, as well as 3′ polyadenylation sequences, that are operable in human cells.

Exemplary isolated polynucleotide molecules include those encoding a FN3 domain comprising an amino acid sequence that is at least 80% identical to the amino acid sequence of SEQ ID NO: 11. In some embodiments, the polynucleotide encodes a FN3 domain comprising an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 11. In some embodiments, the polynucleotide encodes a FN3 domain comprising the amino acid sequence of SEQ ID NO: 11 (Mb_ICAM2_S36). In these embodiments, the polynucleotide may encode one or more amino acid residues of the A, B, C, D, E, and/or F beta-strands that are modified in SEQ ID NO: 11 (from the corresponding wildtype residue) for binding to ICAM-2.

In one embodiment, the exemplary isolated polynucleotide encoding the FN3 domain of SEQ ID NO: 11 (Mb_ICAM2_S36) is a DNA molecule that is codon-optimized for expression in human cells. One such polynucleotide includes the DNA sequence according to SEQ ID NO: 18 as follows:

GTTAGCTCTGTGCCTACCAAGCTGGAAGTGGTGGCTGCTACACCTACCA
GCCTGCTGATCTCCTGGGATGCCCCAGCCGTGACAGTGCTGTACTACTT
CATCACCTACGGCGAGACAGGCCACGGCAGCGCCTACCAGGAGTTCGCC
GTGCCCGGCAGCAAAAGCACCGCCACCATTTCCGGACTGAAGCCTGGCG
TCGACTACACAATCACCGTGTACGCCCTGTGGTACAAGGGCATCACCAG
CCCCATCTCTATCAACTATAGAACCTAA

This polynucleotide can be coupled to any of a variety of promoter and enhancer sequences, as well as 3′ polyadenylation sequences, that are operable in human cells.

Exemplary isolated polynucleotide molecules include those encoding a FN3 domain comprising an amino acid sequence that is at least 80% identical to the amino acid sequence of SEQ ID NO: 12. In some embodiments, the polynucleotide encodes a FN3 domain comprising an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 12. In some embodiments, the polynucleotide encodes a FN3 domain comprising the amino acid sequence of SEQ ID NO: 12 (Mb_ICAM2_S38). In these embodiments, the polynucleotide may encode one or more amino acid residues of the A, B, C, D, E, and/or F beta-strands that are modified in SEQ ID NO: 12 (from the corresponding wildtype residue) for binding to ICAM-2.

In one embodiment, the exemplary isolated polynucleotide encoding the FN3 domain of SEQ ID NO: 12 (Mb_ICAM2_S38) is a DNA molecule that is codon-optimized for expression in human cells. One such polynucleotide includes the DNA sequence according to SEQ ID NO: 19 as follows:

GTTTCTAGCGTGCCCACCAAGCTGGAAGTGGTGGCCGCTACACCTACCA
GCCTGCTGATCAGCTGGGATGCCCCTGCTGTGACCGTGCTGTACTACTT
CATCACATACGGCGAGACAGGCCCTGCCAGCTACGGCGCCCAGGAGTTC
GCCGTGCCAGGCAGCAAGTCCACCGCCACAATTTCTGGCCTGAAACCTG
GAGTGGACTACACCATCACCGTCTACGCCATCTCCAACAAGTGGAACTA
CAGCCCCATCAGCATCAACTATAGAACCTAA

This polynucleotide can be coupled to any of a variety of promoter and enhancer sequences, as well as 3′ polyadenylation sequences, that are operable in human cells.

Exemplary isolated polynucleotide molecules include those encoding a FN3 domain comprising an amino acid sequence that is at least 80% identical to the amino acid sequence of SEQ ID NO: 13. In some embodiments, the polynucleotide encodes a FN3 domain comprising an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to an amino acid sequence of SEQ ID NO: 13. In some embodiments, the polynucleotide encodes a FN3 domain comprising the amino acid sequence of SEQ ID NO: 13 (Mb_ICAM2_S40). In these embodiments, the polynucleotide may encode one or more amino acid residues of the A, B, C, D, E, and/or F beta-strands that are modified in SEQ ID NO: 13 (from the corresponding wildtype residue) for binding to ICAM-2.

In one embodiment, the exemplary isolated polynucleotide encoding the FN3 domain of SEQ ID NO: 13 (Mb_ICAM2_S40) is a DNA molecule that is codon-optimized for expression in human cells. One such polynucleotide includes the DNA sequence according to SEQ ID NO: 20 as follows:

GTGTCCAGCGTGCCCACCAAGCTGGAAGTGGTCGCCGCTACCCCTACCA
GCCTGCTGATCAGCTGGGATGCCCCTGCTGTTACAGTGCTGTACTACTT
CATCACCTACGGCGAGACAGGCGGCAACAGCCCTGTGCAGGAGTTCGCC
GTGCCAGGCAGCAAGTCTACAGCCACAATCAGCGGACTGAAGCCTGGCG
TGGACTACACCATTACCGTGTACGCCCTGAGCTCTAAATGGCGGTACAG
CCCCATCTCCATCAACTATAGAACCTAA

This polynucleotide can be coupled to any of a variety of promoter and enhancer sequences, as well as 3′ polyadenylation sequences, that are operable in human cells.

The polynucleotides of the disclosure may be produced by chemical synthesis such as solid phase polynucleotide synthesis on an automated polynucleotide synthesizer and assembled into complete single or double stranded molecules. Alternatively, the polynucleotides of the disclosure may be produced by other techniques such as PCR followed by routine cloning. Techniques for producing or obtaining polynucleotides of a given known sequence are well known in the art.

The polynucleotides described herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Exemplary constitutive promoter sequences operable in human cells include, without limitation, an EF1 alpha promoter, for example the EF1 alphaS promoter; the PGK promoter; the CMV or SV40 viral promoters; the GAG promoter; the UBC promoter. Other constitutive promoters can also be used (see Qin et al., “Systematic Comparison of Constitutive Promoters and the Doxycycline-Inducible Promoter,” PLOS One 5(5): e10611 (2010), which is hereby incorporated by reference in its entirety). The constitutive promoters can be rendered inducible using, e.g., a transcriptional suppression domain (tTS) adjacent to a high-affinity tTS-binding site (tetO), such that expression is suppressed in the absence of doxycycline but restored in the presence of doxycycline.

Another embodiment of the disclosure is a vector comprising at least one or more of the polynucleotides and fusion constructs as described herein. Such vectors may be plasmid vectors, viral vectors, vectors for baculovirus expression, transposon-based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

In any embodiment, the vector comprising the polynucleotide encoding the ICAM-2 binding polypeptide or fusion construct is a viral vector. Suitable viral vectors include, without limitation, lentiviral vector, an adeno-associated viral vector, vaccinia vector, and a retroviral vector.

Another embodiment of the present disclosure is a host cell comprising the above-described vectors. The ICAM-2 binding molecules and/or ICAM-2 binding peptide conjugates disclosed herein can be optionally produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21 (DE3), BL21-GOLD (DE3), XL1-Blue, JM109, HMS174, HMS174 (DE3), and any of the natural or engineered E. coli spp, Klebsiella spp., or Pseudomonas spp strains.

Mammalian expression systems are generally the preferred platform for manufacturing biopharmaceuticals, as these cell lines are able to produce large, complex proteins with post-translational modifications similar to those produced in humans. Moreover, in the case of mammalian cell lines, most proteins can be secreted rather than requiring cell lysis to extract with subsequent protein refolding (as is the case with bacteria/prokaryotes).

Human cell lines have the ability to produce proteins most similar to those synthesized naturally in humans, which may be an advantage compared with other mammalian expression systems. In particular, the structure, number and location of post-translational N-glycans can affect the biologic activity, protein stability, clearance rate and immunogenicity of biotherapeutic proteins. Exemplary human cell lines that can be used to recombinantly express the ICAM-2 binding molecules include, without limitation, HEK293, fibrosarcoma HT-1080, PER.C6, HKB-11, CAP, and HuH-7 human cell lines.

As noted above, the ICAM-2 binding molecules and/or ICAM-2 binding peptide conjugates, when recombinantly expressed, can then be purified using routine techniques that are well known in the art.

### Pharmaceutical Compositions

A further aspect relates to compositions, including pharmaceutical compositions, that include the disclosed ICAM-2 binding polypeptide, the ICAM-2 binding peptide conjugate, or the isolated polynucleotide or vector; and a pharmaceutically acceptable carrier.

In one embodiment where the ICAM-2 binding polypeptide is present (i.e., in unconjugated form), the composition may include a pharmaceutically active agent, which is one of the pharmaceutically active moieties as described above in unconjugated form.

In another embodiment, the ICAM-2 binding peptide conjugate is present in the composition.

The formulation of pharmaceutically active ingredients with pharmaceutically acceptable carriers is known in the art, e.g., Remington: The Science and Practice of Pharmacy (e.g. 21st edition (2005), and any later editions), which is hereby incorporated by reference in its entirety. Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the invention.

As used herein, the terms “pharmaceutically acceptable carrier” and “pharmaceutically acceptable excipient” (e.g., additives such as diluents, immunostimulants, adjuvants, antioxidants, preservatives and solubilizing agents) are non-toxic to the subject administered the composition at the dosages and concentrations employed.

Examples of pharmaceutically acceptable carriers include water, e.g., buffered with phosphate, citrate and another organic acid, as well as normal saline (about 0.9% NaCl). Representative examples of pharmaceutically acceptable excipients that may be useful in the present disclosure include antioxidants such as ascorbic acid; low molecular weight (less than about 10 residues) polypeptides; hydrophilic polymers such as polyvinylpyrrolidone, dextran, or polyethylene glycol (PEG); amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants.

In any embodiment, the pharmaceutical composition as described herein is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

To improve patient tolerance to administration, the pharmaceutical composition preferably has a pH of about 6 to about 8, preferably about 6.5 to about 7.4. Typically, sodium hydroxide and hydrochloric acid are added as necessary to adjust the pH.

The pharmaceutical composition suitably includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

The composition may also include solubilizing agents, preservatives, stabilizers, emulsifiers, and the like.

Effective amounts of the ICAM-2 binding polypeptide or conjugate will depend on the nature of use, including the nature of the condition which is being treated. By way of example only, suitable ICAM-2 binding polypeptide or conjugate concentrations may range from about 1 μM to about 10 mM, preferably about 10 μM to about 5 mM, about 50 μM to about 2 mM, or about 100 μM to about 1 mM. The volume of the composition administered, and thus, dosage of the peptide administered can be adjusted by one of skill in the art to achieve optimized results. By way of example, 250 μg to 5000 μg, such as 250 μg to 4000 μg or 250 μg to 2000 μg, can be administered per day, repeated periodically as needed, e.g., every other or every third day, once weekly, every other week, etc. This can be adjusted lower to identify the minimal effective dose, or tailored higher or lower according to optimize treatment efficacy.

In certain embodiments, the pharmaceutical composition is formulated as an injectable which can be injected, for example, via an injection device (e.g., a syringe or an infusion pump). By way of example, injectable compositions can be administered intravenously, intradermally, intramuscularly, intraperitoneally, by implantation, by intracavitary or intravesical instillation, intraarterially, intralesionally, peritumorally, intratumorally, or by introduction into one or more lymph nodes. In certain embodiments, administration is carried out intralesionally, intratumorally, intradermally, or peritumorally.

In any embodiment, the pharmaceutical composition as described herein is a solid formulation, e.g., a freeze-dried or spray-dried composition, which can be used as is, or whereto a healthcare professional may add solvents and/or diluents prior to use.

The dosage forms of the pharmaceutical composition may be immediate release, in which case they can comprise a water-soluble or dispersible carrier (as described above), or they can be delayed release, sustained release, or modified release, in which case they can comprise water-insoluble polymers that regulate the rate of dissolution of the dosage form under the skin.

By way of example, targeted delivery of therapeutics to vascular endothelial cells may be particularly effective in suppressing immune-mediated injuries to a donor organ (e.g., heart, lung, liver, kidney) or tissue without causing systemic toxicity to the recipient patient. Targeted delivery of other therapeutics, such as immunostimulants, anti-angiogenic agents, thermoablative nanoparticles and/or chemotherapeutic agents, can also be delivered to endothelial cells associated with neovascularized primary or secondary tumors.

### Therapeutic Uses

The ICAM-2 binding molecules and/or ICAM-2 binding peptide conjugates possess a number of uses, and can be administered to patients in need of a particular treatment as described herein.

Although primarily indicated in the therapeutic (including the curative and/or restorative) treatment of one or more conditions or disease states, the disclosed ICAM-2 binding molecules and/or ICAM-2 binding peptide conjugates may be useful in palliative and/or diagnostic treatment (e.g. during diagnostic workup if a condition is suspected), as well as the prophylactic treatment (by which we include preventing and/or abrogating deterioration and/or worsening of a condition) intended for treatment.

Exemplary patients to be treated in accordance with the present disclosure include individual “subjects” identified above, including veterinary and human patients. The patient to be treated in accordance with the present invention can be a pediatric, juvenile, adult, or geriatric patient.

One aspect relates to a method of inhibiting transplant organ rejection. This method includes the step of administering to a recipient of a donor organ or tissue an effective amount of the ICAM-2 binding peptide conjugate of the invention, wherein the pharmaceutically active moiety is an immunosuppressant agent. As a consequence of administering the ICAM-2 binding peptide-immunosuppressant agent conjugate, the ICAM-2 binding peptide can deliver the conjugate to ICAM-2 expressing tissues where the immunosuppressant agent is effective to suppress rejection of the donor organ or tissue.

It is contemplated that the ICAM-2 binding peptide-immunosuppressant agent conjugate, or pharmaceutical composition containing the same, will be administered repeatedly over the life of the transplant recipient to blunt the immune response and prevent immune-rejection. By administering the ICAM-2 binding peptide-immunosuppressant agent conjugate, or pharmaceutical composition containing the same, it may be possible to eliminate or minimize the systemic administration of immunosuppressant agents independent of the disclosed conjugates.

Another aspect relates to a method of treating hypertension. This method includes the step of administering to an individual having hypertension an effective amount of the ICAM-2 binding peptide conjugate according to the second aspect, wherein the pharmaceutically active moiety is an anti-hypertensive agent, and the administering is effective to treat hypertension. As a consequence of administering the ICAM-2 binding peptide-antihypertensive agent conjugate, the ICAM-2 binding peptide can deliver the conjugate to ICAM-2 expressing tissues where the antihypertensive agent is effective to achieve an antihypertensive effect.

It is contemplated that the ICAM-2 binding peptide-antihypertensive agent conjugate, or pharmaceutical composition containing the same, will be administered repeatedly while the patient displays symptoms of hypertension (elevated blood pressure) in the absence of treatment. By administering the ICAM-2 binding peptide-antihypertensive agent conjugate, or pharmaceutical composition containing the same, it may be possible to eliminate or minimize the systemic administration of antihypertensive agents independent of the disclosed conjugates.

Another aspect relates to a method of treating cancer using an ICAM-2 binding peptide conjugate as disclosed herein.

The cancer (and cancer cells) to be treated in accordance with these aspects can be present in a solid tumor, present as a metastatic cell, or present in a heterogenous population of cells that includes both cancerous and noncancerous cells. Exemplary cancer conditions include, without limitation, cancers or neoplastic disorders of the brain and CNS (glioma, malignant glioma, glioblastoma, astrocytoma, multiforme astrocytic gliomas, medulloblastoma, craniopharyngioma, ependymoma, pinealoma, hemangioblastoma, acoustic neuroma, oligodendroglioma, meningioma), pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), lymph node cancer, lung (Adenocarcinoma, Oat Cell, Non-small Cell, Small Cell, Squamous Cell, Mesothelioma), skin (melanoma, basal cell, squamous cell, Kapsosi's Sarcoma), bone cancer (Ewing's Sarcoma, Osteosarcoma, Chondrosarcoma), head and neck (laryngeal, pharyngeal, and esophageal cancers), oral (jaw, salivary gland, throat, thyroid, tongue, and tonsil cancers), eye, gynecological (Cervical, Endrometrial, Fallopian, Ovarian, Uterine, Vaginal, and Vulvar), genitourinary (Adrenal, bladder, kidney, penile, prostate, testicular, and urinary cancers), and gastrointestinal (appendix, bile duct (extrahepatic bile duct), colon, gallbladder, gastric, intestinal, liver, pancreatic, rectal, and stomach cancers).

According to one embodiment, the method of treatment includes the step of administering to a cancer patient an effective amount of the ICAM-2 binding peptide conjugate, where the pharmaceutically active moiety is an immunostimulant, anti-angiogenic agent, or chemotherapeutic agent. The administered conjugate has the effect of concentrating the immunostimulant, anti-angiogenic agent, or chemotherapeutic agent at a site proximate where the cancerous cells reside, thereby promoting the effective treatment of the cancer.

According to one embodiment, the method of treatment includes the steps of administering to a cancer patient an effective amount of the ICAM-2 binding peptide conjugate, wherein the conjugate includes a thermo-ablative agent; and then exposing the cancer patient to energy suitable to cause localized heating of the thermo-ablative agent at the site of primary and/or secondary tumors to treat the cancer. For example, following administration and concentration of the conjugates at tumor-containing regions of a patient's body, such tumor-containing regions can be exposed to near infrared light, thereby causing thermal heating of the thermoablative nanoparticle and destruction of cancer cells proximate the conjugate.

It is also contemplated that the conjugates as described herein can be used in conjunction with one or more of chemotherapeutic agents, immunotherapeutic agents, or radiotherapeutic agents, as well as surgical intervention. Thus, a chemotherapeutic agent, an immunotherapeutic agent, or a radiotherapeutic agent can be administered to a patient before or after treatment with the conjugates of the present invention. Alternatively, surgical resection of a tumor can be carried out before or after treatment with the conjugates of the present invention.

### EXAMPLES

The examples below are intended to exemplify the practice of embodiments of the disclosure but are by no means intended to limit the scope thereof.

### Example 1-Development of Monobodies Selective to Human ICAM-2

Human ICAM-2 extracellular region (UniProt ID P13598; residues 25-223) was expressed C-terminally fused to His6 and Avi-tags from EXPI293 cells (Thermo Fisher). The protein was purified and biochemically biotinylated using the BirA enzyme. Using this protein as an antigen, selection of monobody libraries was performed using phage display and yeast display by following published procedures (Teng et al., “Selective and Noncovalent Targeting of RAS Mutants for Inhibition and Degradation,” Nat. Commun. 12 (1): 2656 (2021); Koide et al., “Teaching an Old Scaffold New Tricks: Monobodies Constructed Using Alternative Surfaces of the FN3 Scaffold,” J. Mol. Biol. 415 (2): 393-405 (2012), each of which is hereby incorporated by reference in its entirety).

Using these procedures, four monobody clones were identified that bound to ICAM-2 with K_{D} values in the nanomolar range, as assayed using yeast display. Subsequently, these monobodies were produced as purified proteins N-terminally fused to His6 and Avi tags. These proteins are soluble and monomeric. Biolayer interferometry measurements showed that these monobodies bound to ICAM-2 with K_{D} values in the single nanomolar range.

These monobodies can be further modified to facilitate a particular application. For example, a single Cys residue can be introduced for site-specific chemical reaction for immobilization to nanomaterials and for conjugating a chemical moiety (e.g., fluorescent dye) or drug compounds. They can be fused with another protein such as fluorescent proteins, antibodies, and enzymes.

Amino acid sequences of monobodies binding to human ICAM-2 include:

(Mb_ICAM2_S32)
SEQ ID NO: 10
VSSVPTKLEVVAATPTSLLISWDAPAVTVLYYFITYGETGGNSPVQEFA
VPGSKSTATISGLKPGVDYTITVYAINQYWKYSPISINYRT
(Mb_ICAM2_S36)
SEQ ID NO: 11
VSSVPTKLEVVAATPTSLLISWDAPAVTVLYYFITYGETGHGSAYQEFA
VPGSKSTATISGLKPGVDYTITVYALWYKGITSPISINYRT
(Mb_ICAM2_S38)
SEQ ID NO: 12
VSSVPTKLEVVAATPTSLLISWDAPAVTVLYYFITYGETGPASYGAQEF
AVPGSKSTATISGLKPGVDYTITVYAISNKWKYSPISINYRT
(Mb_ICAM2_S40)
SEQ ID NO: 13
VSSVPTKLEVVAATPTSLLISWDAPAVTVLYYFITYGETGGNSPVQEFA
VPGSKSTATISGLKPGVDYTITVYALSSKWRYSPISINYRT

FIG. 3 is a Clustal Omega (version 1.2.4) alignment of the four monobodies selected against human ICAM-2. Variations within the CD and FG loop sequences are shown.

### Example 2-Affinity Measurements of Human ICAM-2-Binding Monobodies

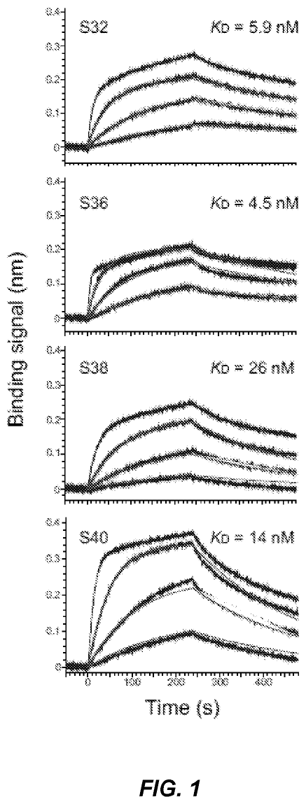

The four monobody samples identified in Example 1 were produced as purified proteins with N-terminal tags containing His6 and Avi-tag, followed by enzymatic biotinylation using the BirA enzyme. The proteins were immobilized on streptavidin-coated biolayer interferometry (BLI) tips using an Octet instrument (Sartorius) and their interaction with human ICAM-2 was monitored with the instrument (FIG. 1). These monobodies had strong binding with the dissociation constant (K_{D}) values ranging from 4.5 to 26 nM.

FIG. 1 depicts BLI sensorgrams. The global fitting of the 1:1 binding model was performed on the data after excluding the highest concentration sensorgrams, because the association rates in the highest concentration data are too high for accurate fitting. The K_{D} values from the global fitting are shown.

### Example 3-Deep Mutational Scanning of SEQ ID NO: 13 (Mb_ICAM2_S40)

To more comprehensively define residues of Mb_ICAM2_S40 (referred to as S40 hereafter) important for binding to ICAM-2, deep mutational scanning was performed (Fowler et al., “High-resolution Mapping of Protein Sequence-function Relationships,” Nat Methods. 7 (9): 741-6 (2010) (doi: 10.1038/nmeth.1492; PMID: 20711194), which is hereby incorporated by reference in its entirety). Using published crystal structures of monobody-target complexes (Hantschel et al., “Monobodies as Enabling Tools for Structural and Mechanistic Biology,” Curr Opin Struct Biol. 60:167-174 (2020) (doi: 10.1016/j.sbi.2020.01.015; PMID: 32145686), which is hereby incorporated by reference in its entirety) as guides, a total of 28 residues in S40 (SEQ ID NO: 13) that are likely to be located in or near the binding interface were selected, and a yeast-display library was generated in which one of these positions was diversified to all 20 amino acids at a time. The library was sorted into four classes: first, clones that exhibit a binding profile similar to the wild-type S40 when measured with 15 nM ICAM-2; second, those similar to the wild type when measured with 30 nM ICAM-2; third, those similar to the wild type when measured with 100 nM ICAM-2; and fourth, those that do not show binding when measured with 100 nM ICAM-2 (“nonbinders”). These pools were subjected to deep sequencing and the numbers of reads for individual mutations were determined. As expected, there are high degrees of overlap among the pools selected for binding to ICAM-2, and there is little overlap between these binding pools and the nonbinder pool. The table below summarizes the results and indicate substitutions that maintain binding to 100 nM ICAM-2.

(Mb_ICAM2_S40)
SEQ ID NO: 13
VSSVPTKLEVVAATPTSLLISWDAPAVTVLYYFITYGETGGNSPVQEFA
VPGSKSTATISGLKPGVDYTITVYALSSKWRYSPISINYRT

TABLE 2
Deep Mutational Scanning of SEQ ID NO: 13

| | strand/ | wild-type | | |
|---|---|---|---|---|
| Position | loop | residue | Permissible mutations (100 nM) | Consensus amino acids |
| 27 | BC | V | A, D, F, G, H, I, K, L, M, N, P, S, T, Y | A, D, F, G, H, I, K, L, M, N, P, S, T, V, Y |
| 28 | BC | T | A, D, G, H, I, K, L, N, P, Q, R, S, V, Y | A, D, G, H, I, K, L, N, P, Q, R, S, T, V, Y |
| 30 | BC | L | F, M, R | F, L, M, R |
| 31 | C | Y | (none) | Y |
| 33 | C | F | M, S, Y | F, M, S, Y |
| 35 | C | T | A, D, E, G, H, I, K, L, M, N, Q, R, S, V, Y | A, D, E, G, H, I, K, L, M, N, Q, R, S, T, V, Y |
| 37 | C | G | A, D, E, F, H, I, L, M, N, Q, R, S, T, V, W | A, D, E, F, G, H, I, L, M, N, Q, R, S, T, V, W |
| 38 | C | E | A, D, H, I, K, L, M, N, P, Q, R, S, V, W, Y | A, D, E, H, I, K, L, M, N, P, Q, R, S, V, W, Y |
| 41 | CD | G | A, D, E, F, H, K, L, M, N, P, Q, R, S, T, V, W | A, D, E, F, G, H, K, L, M, N, P, Q, R, S, T, V, W |
| 42 | CD | N | A, D, E, F, G, H, I, K, L, M, P, Q, R, T, V, W, Y | A, D, E, F, G, H, I, K, L, M, N, P, Q, R, T, V, W, Y |
| 43 | CD | S | A, E, F, G, H, I, K, L, M, N, P, Q, R, T, V, W, Y | A, E, F, G, H, I, K, L, M, N, P, Q, R, S, T, V, W, Y |
| 44 | CD | P | A, D, E, F, G, H, I, K, L, M, N, Q, R, S, T, V, W, Y | A, D, E, F, G, H, I, K, L, M, N, P, Q, R, S, T, V, W, Y |
| 45 | D | V | A, D, E, F, G, H, I, L, M, N, P, Q, R, S, T, W, Y | A, D, E, F, G, H, I, L, M, N, P, Q, R, S, T, W, Y |
| 46 | D | Q | A, D, E, H, I, L, M, N, P, S, T, V, W | A, D, E, H, I, L, M, N, P, Q, S, T, V, W |
| 47 | D | E | (none) | E |
| 49 | D | A | V | A, V |
| 71 | F | T | A, D, E, F, G, H, K, L, M, N, P, Q, R, S, V, W, Y | A, D, E, F, G, H, K, L, M, N, P, Q, R, S, T, V, W, Y |
| 73 | F | Y | A, C, E, F, H, N, T, W | A, C, E, F, H, N, T, W, Y |
| 74 | F | A | G | A, G |
| 75 | F | L | M, R, V | L, M, R, V |
| 76 | FG | S | D, G, L, N, T | D, G, L, N, S, T |
| 77 | FG | S | A, D, F, G, H, K, M, P, Q, R, T, V, W, Y | A, D, F, G, H, K, M, P, Q, R, S, T, V, W, Y |
| 78 | FG | K | D, I, N, Q, R, S, T | D, I, K, N, Q, R, S, T |
| 79 | FG | W | F, I, Y | F, I, Y, W |
| 80 | FG | R | K, L, M | K, L, M, R |
| 81 | FG | Y | F, H | F, H, Y |
| 82 | FG | S | A, E, F, G, H, I, M, P, R, T, W, Y | A, E, F, G, H, I, M, P, R, S, T, W, Y |
| 83 | G | P | D, H, T | D, H, P, T |

The combination of any two or more substitutions listed in Table 2 are contemplated. These include two or more substitutions appearing in the same structural region (e.g., BC loop, CD loop, FG loop, C strand, D strand, or F strand) as well as two or more substitutions in different locations. Non-limiting examples of the latter include (i) one or more substitution at positions 27, 28 or 30 of the BC loop in combination with one or more substitution at positions 41-44 of the CD loop; (ii) one or more substitution at positions 27, 28 or 30 of the BC loop in combination with one or more substitution at positions 76-82 of the FG loop; (iii) one or more substitution at positions 41-44 of the CD loop in combination with one or more substitution at positions 76-82 of the FG loop; (iv) one or more substitution at positions 27, 28 or 30 of the BC loop in combination with one or more substitution at positions 41-44 of the CD loop and one or more substitution at positions 76-82 of the FG loop; (v) one or more substitution at positions 27, 28 or 30 of the BC loop in combination with one or more substitution in the C, D, F, or G strands as shown in Table 2; (vi) one or more substitution at positions 41-44 of the CD loop in combination with one or more substitution in the C, D, F, or G strands as shown in Table 2; and (vii) one or more substitution at positions 76-82 of the FG loop in combination with one or more substitution in the C, D, F, or G strands as shown in Table 2.

### Example 4-Detection of Human ICAM-2 on the Surface of Human Cells

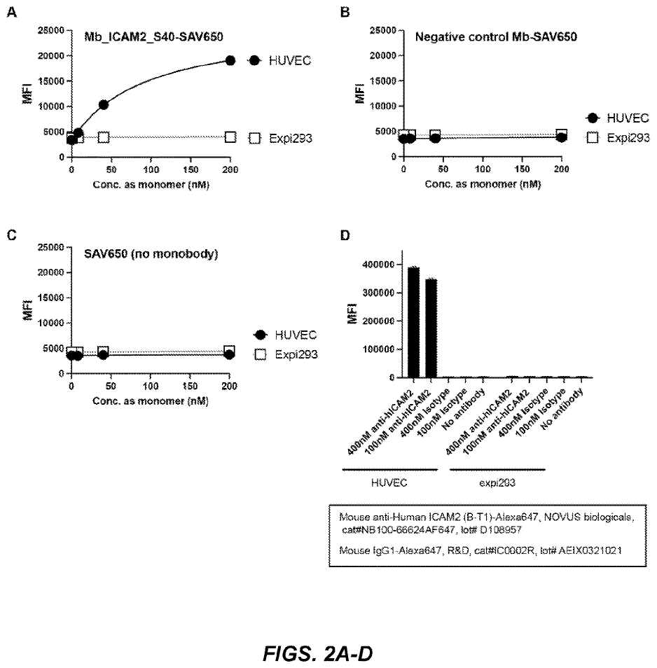

Whether Mb_ICAM2_S40 detected endogenous ICAM-2 molecules expressed on the surface of human cells was tested. Biotinylated Mb_ICAM2_S40 was bound to streptavidin DyLight 650 and reacted with Primary Umbilical Vein Endothelial Cells (HUVEC) that express ICAM-2 or with Expi293 cells (Thermo Fisher) that do not express ICAM-2 (as a negative control) and bound monobody was detected using flow cytometry (FIG. 2A). Mb_ICAM2_S40, but not a negative control monobody (FIG. 2B) or streptavidin DyLight 650 without bound monobody (FIG. 2C), showed dose-dependent signals to HUVEC but not Expi293 cells, confirming that Mb_ICAM2_S40 is capable of detecting endogenous human ICAM-2. The expression of ICAM-2 in HUVEC but not in Expi293 was confirmed using a commercially available anti-hICAM-2 antibody (FIG. 2D).

The negative control monobody has the amino acid sequence of SEQ ID NO: 34 as follows:

VSSVPTKLEVVAATPTSLLISWDASSSSVSYYRITYGETGGNSPVQEFT
VPGSKSTATISGLKPGVDYTITVYASSSSSSSSSSSKPISINYRT

This sequence was published in the supplementary information of Wallen et al., “Inhibition of RAS-driven Signaling and Tumorigenesis with a Pan-RAS Monobody Targeting the Switch I/II Pocket,” Proc. Nat'l Acad. Sci USA 119 (43): e2204481119 (doi: 10.1073/pnas.2204481119) (PMID: 36252024), which is hereby incorporated by reference in its entirety.

### Example 5-Development of Monobodies Selective to Pig ICAM-2

To enable studies using pig organs as model systems, monobodies selective to pig ICAM-2 (pICAM-2) were also developed. pICAM-2 extracellular region (UniProt ID Q6VY03; residues 24-250) was expressed C-terminally fused to His6 and Avi-tags and purified, essentially following the methods described for human ICAM-2 in Example 1. Likewise, monobody library sorting using phage display and yeast display was performed as described in Example 1. The following monobodies that bound to pICAM-2 were identified using yeast display:

Mb_pICAM2_L1
(SEQ ID NO: 21)
VSSVPTKLEVVAATPTSLLISWDAGYWSSVAYYRITYGETGGNSPVQEF
TVPGSSSTATISGLKPGVDYTITVYAMTSGYSWYSPISINYRT
Mb_pICAM2_L2
(SEQ ID NO: 22)
VSSVPTKLEVVAATPTSLLISWDAEYWVSVMYYRITYGETGGNSPVQEF
TVPYSSYTATISGLKPGVDYTITVYAQTSMYSWYSPISINYRT
Mb_pICAM2_L3
(SEQ ID NO: 23)
VSSVPTKLEVVAATPTSLLISWDAGYWSSVAYYRITYGETGGNSPVQEF
TVPSSSSTATISGLKPGVDYTITVYATTSQYSWYSPISINYRT
Mb_pICAM2_L5
(SEQ ID NO: 24)
VSSVPTKLEVVAATPTSLLISWDAGYWSSVAYYRITYGETGGNSPVQEF
TVPYYSYTATISGLKPGVDYTITVYATTSQYSWYSPISINYRT
Mb_pICAM2_L6
(SEQ ID NO: 25)
VSSVPTKLEVVAATPTSLLISWDAGYWSSVAYYRITYGETGGNSPVQEF
TVPSSSSTATISGLKPGVDYTITVYAQTSMYSWYSPISINYRT
Mb_pICAM2_L7
(SEQ ID NO: 26)
VSSVPTKLEVVAATPTSLLISWDAEYYSSVHYYRITYGETGGNSPVQEF
TVPGSSSTATISGLKPGVDYTITVYAMTSGYSWYSPISINYRT
Mb_pICAM2_L10
(SEQ ID NO: 27)
VSSVPTKLEVVAATPTSLLISWDAEYWVSVMYYRITYGETGGNSPVQEF
TVPYSSYTATISGLKPGVDYTITVYATTSQYSWYSPISINYRT
Mb_pICAM2_L11
(SEQ ID NO: 28)
VSSVPTKLEVVAATPTSLLISWDAGYWSSVAYYRITYGETGGNSPVQEF
TVPSSSSTATISGLKPGVDYTITVYAMTSGYSWYSPISINYRT
Mb_pICAM2_L13
(SEQ ID NO: 29)
VSSVPTKLEVVAATPTSLLISWDAGYWSSVAYYRITYGETGGNSPVQEF
TVPSSSSTATISGLKPGVDYTITVYAQTSYYSWYSPISINYRT
Mb_pICAM2_L14
(SEQ ID NO: 30)
VSSVPTKLEVVAATPTSLLISWDAGYWSSVSYYRITYGETGGNSPVQEF
TVPGSSYTATISGLKPGVDYTITVYAQTSYYSWYSPISINYRT
Mb_pICAM2_L22
(SEQ ID NO: 31)
VSSVPTKLEVVAATPTSLLISWDAGYWSSVAYYRITYGETGGNSPVQEF
TVPGSSYTATISGLKPGVDYTITVYAKTSDYSWYSPISINYRT
Mb_pICAM2_L27
(SEQ ID NO: 32)
VSSVPTKLEVVAATPTSLLISWDAGYWSSVAYYRITYGETGGNSPVQEF
TVPGSYSTATISGLKPGVDYTITVYAQTSMYSWYSPISINYRT
Mb_pICAM2_L32
(SEQ ID NO: 33)
VSSVPTKLEVVAATPTSLLISWDAEEWSSVSYYRITYGETGGNSPVQEF
TVPYYSSTATISGLKPGVDYTITVYATTSQYSWYSPISINYRT

FIG. 4 is a Clustal Omega (version 1.2.4) alignment of the thirteen monobodies selected against pig ICAM-2. Variations within the BC, DE, and FG loop sequences are shown.

Further modification of the pICAM-2 monobodies using the substitutions shown in Table 2 are also contemplated, and these include the combination of any two or more substitutions listed in Table 2. These include two or more substitutions appearing in the same structural region (e.g., BC loop, CD loop, FG loop, C strand, D strand, or F strand) as well as two or more substitutions in different locations. Non-limiting examples of the latter include (i) one or more substitution at positions 27, 28 or 30 of the BC loop in combination with one or more substitution at positions 41-44 of the CD loop; (ii) one or more substitution at positions 27, 28 or 30 of the BC loop in combination with one or more substitution at positions 76-82 of the FG loop; (iii) one or more substitution at positions 41-44 of the CD loop in combination with one or more substitution at positions 76-82 of the FG loop; (iv) one or more substitution at positions 27, 28 or 30 of the BC loop in combination with one or more substitution at positions 41-44 of the CD loop and one or more substitution at positions 76-82 of the FG loop; (v) one or more substitution at positions 27, 28 or 30 of the BC loop in combination with one or more substitution in the C, D, F, or G strands as shown in Table 2; (vi) one or more substitution at positions 41-44 of the CD loop in combination with one or more substitution in the C, D, F, or G strands as shown in Table 2; and (vii) one or more substitution at positions 76-82 of the FG loop in combination with one or more substitution in the C, D, F, or G strands as shown in Table 2.

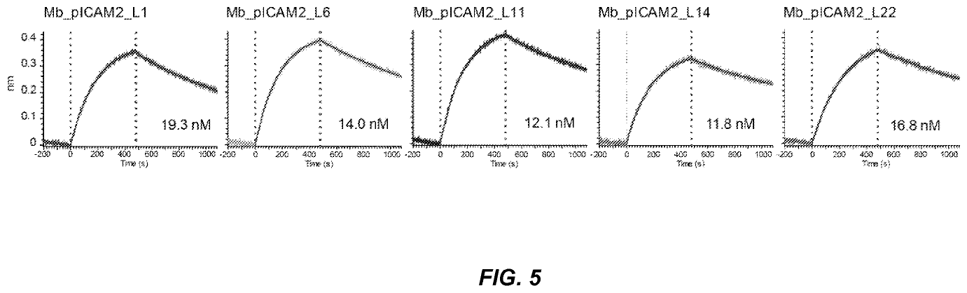

Biolayer interferometry (BLI) for five of the monobodies (Mb_pICAM2_L1, Mb_pICAM2_L6, Mb_pICAM2_L11, Mb_pICAM2_L14, and Mb_pICAM2_L22) was carried out using the procedures and equipment described above (FIG. 5). These monobodies had strong binding with the dissociation constant (K_{D}) values ranging from 11.8 to 19.3 nM.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed:

1. An Intercellular Adhesion Molecule 2 (ICAM-2) binding polypeptide, said binding polypeptide comprising a fibronectin type III (FN3) domain, said FN3 domain comprising at least one modified loop amino acid sequence, wherein said at least one modified loop sequence enables binding to ICAM-2.

2. The binding polypeptide of claim 1, wherein said at least one modified loop sequence is a modified CD loop amino acid sequence, a modified FG loop amino acid sequence, or a combination thereof.

3. The binding polypeptide of claim 1, wherein said modified CD loop amino acid sequence comprises an amino acid sequence of T-G-(P/H)-(G/A)-S-(Y/A)-X-(Y/A) (SEQ ID NO: 2), where X is optional and can be Gly (G).

4. The binding polypeptide of claim 1 or 3, wherein said modified CD loop amino acid sequence is selected from any one of SEQ ID NO: 3 or 4:

| CD Loop Amino Acid Sequence | SEQ ID NO: |
|---|---|
| TGHGSAY | 3 |
| TGPASYGA | 4 |

5. The binding polypeptide of claim 1, wherein said modified FG loop amino acid sequence comprises an amino acid sequence of (Y/K)-W-(K/R)—Y—S-P (SEQ ID NO: 5).

6. The binding polypeptide of claim 1 or 5, wherein said modified FG loop amino acid sequence is selected from any one of SEQ ID NOs: 6-9:

| FG Loop Amino Acid Sequence | SEQ ID NO: |
|---|---|
| NQYWKYSP | 6 |
| LWYKGITSP | 7 |
| ISNKWKYSP | 8 |
| LSSKWRYSP | 9 |

7. The binding polypeptide of any one of claims 1-6, wherein said at least one modified loop sequence further comprises a modified BC loop amino acid sequence, a modified DE loop amino acid sequence, or both.

8. The binding polypeptide of any one of claims 1-4, wherein said binding polypeptide comprises a wildtype AB loop amino acid sequence, a wildtype BC loop amino acid sequence, a wildtype CD loop amino acid sequence, a wildtype DE loop amino acid sequence, a wildtype EF loop amino acid sequence, or any combination thereof.

9. The binding polypeptide of any one of claims 1-8, wherein the FN3 domain is a human fibronectin type III tenth domain (10Fn3) of SEQ ID NO:1 comprising at least one modified loop amino acid sequence.

10. The binding polypeptide of claim 9, wherein the 10Fn3 domain further comprises an amino acid substitution in one or more of the C, D, F, or G beta-strands.

11. The binding polypeptide of claim 10, wherein the amino acid substitution is at one or more residues selected from Y31, R33, E47, T49, Y73, A74, and V75 of SEQ ID NO: 1.

12. The binding polypeptide of claim 10, wherein the amino acid substitution at R33 is selected from the group consisting of R33V, R33D, and R33F.

13. The binding polypeptide of claim 10, wherein the amino acid substitution at E47 is selected from the group consisting of E47T and E47K.

14. The binding polypeptide of claim 10, wherein the amino acid substitution at T49 is selected from the group consisting of T49K and T49A.

15. The binding polypeptide of claim 10, wherein the amino acid substitution at A74 is A74T.

16. The binding polypeptide of claim 9 or 10, further comprising an amino acid substitution at one or more resides selected from D3, R6 and D7 of SEQ ID NO: 1.

17. The binding polypeptide of any one of claims 1-16, wherein the FN3 domain comprises:
(i) a modified FG loop amino acid sequence comprising SEQ ID NO: 6 and a wildtype CD loop amino acid sequence (S32);
(ii) a modified FG loop amino acid sequence comprising SEQ ID NO: 7 and a modified CD loop amino acid sequence comprising SEQ ID NO: 3 (S36);
(iii) a modified FG loop amino acid sequence comprising SEQ ID NO: 8 and a modified CD loop amino acid sequence comprising SEQ ID NO: 4 (S38); or
(iv) a modified FG loop amino acid sequence comprising SEQ ID NO: 9 and a wildtype CD loop amino acid sequence (S40).

18. The binding polypeptide of any one of claims 1-16, wherein the FN3 domain comprises:
(i) a modified FG loop amino acid sequence consisting of SEQ ID NO: 6 and a wildtype CD loop amino acid sequence (S32);
(ii) a modified FG loop amino acid sequence consisting of SEQ ID NO: 7 and a modified CD loop amino acid sequence consisting of SEQ ID NO: 3 (S36);
(iii) a modified FG loop amino acid sequence consisting of SEQ ID NO: 8 and a modified CD loop amino acid sequence consisting of SEQ ID NO: 4 (S38); or
(iv) a modified FG loop amino acid sequence consisting of SEQ ID NO: 9 and a wildtype CD loop amino acid sequence (S40).

19. The binding polypeptide of any one of claims 1-16, wherein the FN3 domain comprises an amino acid sequence that is at least 80% identical to an amino acid sequence selected from the group consisting of SEQ ID NOs: 10-13.

20. The binding polypeptide of any one of claims 1-16, wherein the FN3 domain comprises an amino acid sequence that is at least 90% identical to an amino acid sequence selected from the group consisting of SEQ ID NOs: 10-13.

21. The binding polypeptide of any one of claims 1-16, wherein the FN3 domain comprises an amino acid sequence that is at least 95% identical to an amino acid sequence selected from the group consisting of SEQ ID NOs: 10-13.

22. The binding polypeptide of any one of claims 1-18, wherein the FN3 domain comprises an amino acid sequence selected from the group of SEQ ID NOs: 10-13 as follows:
(Mb_ICAM2_S32)
SEQ ID NO: 10
VSSVPTKLEVVAATPTSLLISWDAPAVTVLYYFITYGETGGNSPVQEF
AVPGSKSTATISGLKPGVDYTITVYAINQYWKYSPISINYRT;
(Mb_ICAM2_S36)
SEQ ID NO: 11
VSSVPTKLEVVAATPTSLLISWDAPAVTVLYYFITYGETGHGSAYQEF
AVPGSKSTATISGLKPGVDYTITVYALWYKGITSPISINYRT;
(Mb_ICAM2_S38)
SEQ ID NO: 12
VSSVPTKLEVVAATPTSLLISWDAPAVTVLYYFITYGETGPASYGAQE
FAVPGSKSTATISGLKPGVDYTITVYAISNKWKYSPISINYRT;
and
(Mb_ICAM2_S40)
SEQ ID NO: 13
VSSVPTKLEVVAATPTSLLISWDAPAVTVLYYFITYGETGGNSPVQEF
AVPGSKSTATISGLKPGVDYTITVYALSSKWRYSPISINYRT.

23. The binding polypeptide of any one of claims 1-22, wherein the FN3 domain comprises a variant of the amino acid sequence of SEQ ID NO: 13 that includes one or more of the substitutions identified in Table 2.

24. The binding polypeptide of any one of claims 1-23, wherein the ICAM-2 is a mammalian ICAM-2.

25. The binding polypeptide of claim 24, wherein the ICAM-2 is a human ICAM-2 or pig ICAM-2.

26. The binding polypeptide of any one of claims 1-25, wherein the polypeptide further comprises a linker sequence tethered to an N-terminal or a C-terminal end of the FN3 domain.

27. The binding polypeptide of claim 26, wherein the linker sequence comprises a cysteine (C) residue.

28. The binding polypeptide of claim 26 or claim 27, wherein the linker sequence comprises the amino acid sequence (D/E) n where n is an integer from 6 to 20.

29. The binding polypeptide of claim 28, wherein the linker sequence comprises a poly-aspartic acid or poly-glutamic acid sequence containing from 6 to 20 residues.

30. An ICAM-2 binding peptide conjugate, said conjugate comprising:
a first portion, said first portion comprising the binding polypeptide of any one of claims 1-29; and
a second portion coupled to said first portion, said second portion selected from a pharmaceutically active moiety, a diagnostic moiety, a half-life extending moiety, a delivery vehicle, a prodrug, a second binding molecule, a polymer, a nanoparticle, and a non-binding protein.

31. The ICAM-2 binding peptide conjugate of claim 30, wherein the second portion is a pharmaceutically active moiety.

32. The ICAM-2 binding peptide conjugate of claim 30, wherein the pharmaceutically active moiety is selected from the group consisting of a small molecule, a nucleic acid molecule, an antibody or antigen binding fragment thereof, an antibody derivative, a protein or polypeptide fragment thereof.

33. The ICAM-2 binding peptide conjugate of claim 32, wherein the nucleic acid is an mRNA molecule, siRNA molecule, shRNA molecule.

34. The ICAM-2 binding peptide conjugate of claim 32, wherein the pharmaceutically active moiety is an anti-inflammatory agent.

35. The ICAM-2 binding peptide conjugate of claim 34, wherein the anti-inflammatory agent is a non-steroidal anti-inflammatory drug (NSAID).

36. The ICAM-2 binding peptide conjugate of claim 35, wherein the NSAID is ibuprofen, naproxen, fenoprofen, ketoprofen, flurbiprofen, oxaprozin, indomethacin, sulindac, etodolac, diclofenac, piroxicam, meloxicam, tenoxicam, droxicam, lomoxicam, isoxicam, mefenamic acid, meclofenamic acid, flufenamic acid, tolfenamic acid, celecoxib, rofecoxib, valdecoxib, parecoxib, lumiracoxib, or etoricoxib.

37. The ICAM-2 binding peptide conjugate of claim 32, wherein the pharmaceutically active moiety is an immunomodulatory (immunostimulating or immunosuppressive) agent.

38. The ICAM-2 binding peptide conjugate of claim 37, wherein the immunomodulatory agent is a steroid.

39. The ICAM-2 binding peptide conjugate of claim 38, wherein the steroid is betamethasone, dexamethasone, flumethasone, methylprednisolone, paramethasone, prednisolone, prednisone, triamcinolone, hydrocortisone or cortisone, alcomethasone dipropionate, amcinonide, betamethasone dipropionate, betamethasone monopropionate, betamethasone 17-valerate, budesonide, budesonide disodium phosphate, ciclomethasone, clobetasol-17-propionate, clobetasone-17-butyrate, cortisone acetate, deprodone propionate, desonide, desoxymethasone, dexamethasone acetate, diflucortolone valerate, diflurasone diacetate, diflucortolone, difluprednate, flumetasone pivalate, flunisolide, fluocinolone acetonide acetate, fluocinonide, fluocortolone, fluocortolone caproate, fluocortolone hexanoate, fluocortolone pivalate, fluormetholone acetate, fluprednidene acetate, fluticasone propionate, halcinonide, halometasone, hydrocortisone acetate, hydrocortisone-17-butyrate, hydrocortisone-17-valerate, medrysone, methylprednisolone acetate, mometasone furoate, parametasone acetate, prednicarbate, prednisolone acetate, prednylidene, rimexolone, tixocortol pivalate, triamcinolone acetonide, triamcinolone alcohol, triamcinolone hexacetonide, betamethasone sodium phosphate, desonide sodium phosphate, dexamethasone sodium phosphate, hydrocortisone sodium phosphate, hydrocortisone sodium succinate, cortisone sodium phosphate, cortisone sodium succinate, methylprednisolone disodium phosphate, methylprednisolone sodium succinate, methylprednisone disodium phosphate, methylprednisone sodium succinate, prednisolone sodium phosphate, prednisolone sodium succinate, prednisone sodium phosphate, prednisone sodium succinate, prednisolamate hydrochloride, triamcinolone acetonide disodium phosphate, or triamcinolone acetonide dipotassium phosphate.

40. The ICAM-2 binding peptide conjugate of claim 37, wherein the immunomodulatory agent is aspirin, methotrexate, sulfasalazine, D-penicillamine, nambumetone, aurothioglucose, auranofin, colloidal gold, cyclosporine, rapamycin, tacrolimus, pimecrolimus, everolimus, sirolimus, tofacitinib, azathioprine, leflunomide, mycophenolate, abatacept, adalimumab, anakinra, certolizumab, etanercept, golimumab, infliximab, ixekizumab, natalizumab, rituximab, secukinumab, tocilizumab, ustekinumab, vedolizumab, basiliximab, or daclizumab, as well as any active binding fragments of the above-listed antibodies.

41. The ICAM-2 binding peptide conjugate of claim 37, wherein the immunomodulatory agent is an interleukin or interferon.

42. The ICAM-2 binding peptide conjugate of claim 31, wherein the pharmaceutically active moiety is an anti-hypertensive agent.

43. The ICAM-2 binding peptide conjugate of claim 42, wherein the anti-hypertensive agent is any one or more of a diuretic, an adrenergic receptor antagonist, an adrenergic receptor agonist, a calcium channel blocker, an Angiotensin-Converting Enzyme (ACE) inhibitor, an angiotensin II receptor antagonist, an aldosterone antagonist, a vasodilator, or a renin inhibitor.

44. The ICAM-2 binding peptide conjugate of claim 43, wherein:
(i) the diuretic is a loop diuretic, such as furosemide, bumetanide, ethacrynic acid, and torsemide;
(ii) the diuretic is a thiazide diuretic, such as epitizide, hydrochlorothiazide, hydroflumethiazide, chlorothiazide, bendroflumethiazide, polythiazide, trichlormethiazide, cyclopenthiazide, methyclothiazide, cyclothiazide, mebutizide, and other benzothiadiazine derivatives; or a thiazide-like diuretic, such as indapamide, chlortalidone, metolazone, quinethazone, clopamide, mefruside, clofenamide, meticrane, xipamide, clorexolone, or fenquizone;
(iii) the diuretic is a potassium-sparing diuretic, such as amiloride, triamterene, eplerenone, benzamil, potassium canrenoate, canrenone, or spironolactone;
(iv) the diuretic is an osmotic diuretic, such as mannitol, glucose, and urea;
(v) the diuretic is a vasopressin receptor antagonist, such as conivaptan, relcovaptan, nelivaptan, lixivaptan, mozavaptan, satavaptan, tolvaptan, or demeclocycline;
(vi) the diuretic is a mercurial diuretics such as mersalyl acid (Mersal), meralluride, mercaptomerin, mercurophylline, merethoxylline procaine, and calomel;
(vii) the diuretic is a xanthine diuretic such as caffeine, theobromine, paraxanthine, or theophylline;
(viii) the diuretic is a carbonic anhydrase inhibitor, such as acetazolamide, methazolamide, dorzolamide, sulfonamide, or topiramate; and
(ix) the diuretic is a diuretic purine, a diuretic steroid, a diuretic sulfonamide derivative, a diuretic uracil derivative, amanozine, arbutin, chlorazanil, etozolin, hydracarbazine, isosorbide, metochalcone, muzolimine, perhexiline, ticrynafen, triamterene, or spironolactone;
(x) the adrenergic receptor antagonist is a beta blocker such as atenolol, metoprolol, nadolol, oxprenolol, pindolol, propranolol, timolol, acebutolol, bisoprolol, esmolol, labetalol, carvedilol, bucindolol, nebivolol, alprenolol; amosulalol, arotinolol, befunolol, betaxolol, bevantolot, bopindolol, bucumolol, bufetolol, bufuralol, bunitrolol, bupranolol, butidrine hydrochloride, butofilolol, carazolol, carteolol, celiprolol, cetamolol, cloranololdilevalol, epanolol, indenolol, levobunolol, mepindolol, metipranolol, moprolol, nadoxolol, nipradilol, penbutolol, practolol, pronethalol, sotalol, sulfinalol, talinolol, tertatolol, tilisolol, toliprolol, or xibenolol;
(xi) the adrenergic receptor antagonist is an alpha blocker such as such as phenoxybenzamine, prazosin, doxazosin, terazosin, trimazosin, phentolamine, amosulalol, arotinolol, dapiprazole, fenspiride, indoramin, labetalol, naftopidil, nicergoline, tamsulosin, tolazoline, reserpine, moxonidine or yohimbine;
(xii) the adrenergic receptor agonist is one of clonidine, methyldopa, guanfacine, methoxamine, methylnorepinephrine, oxymetazoline, phenylephrine, guanabenz, guanoxabenz, guanethidine, xylazine, or tizanidine;
(xiii) the calcium channel blocker is a dihydropyridine such as amlodipine, felodipine, nicardipine, nifedipine, nimodipine, isradipine, nitrendipine, aranidipine, barnidipine, benidipine, cilnidipine, efonidipine, elgodipine, lacidipine, lercanidipine, manidipine, nilvadipine, or nisoldipine;
(xiv) the calcium channel blocker is a non-dihydropyridine such as diltiazem, verapamil, bepridil, clentiazem, fendiline, gallopamil, mibefradil, prenylamine, semotiadil, terodiline, cinnarizine, flunarizine, lidoflazine, lomerizine, bencyclane, etafenone, or perhexiline;
(xv) the ACE inhibitor is a sulfhydryl-containing agent such as captopril or zofenopril;
(xvi) the ACE inhibitor is a dicarboxylate-containing agent such as enalapril, ramipril, quinapril, perindopril, lisinopril, or benazepril;
(xvii) the ACE inhibitor is a phosphonate-containing agent such as fosinopril or ceronapril;
(xviii) the ACE inhibitor is a naturally occurring ACE inhibitor such as casokinins, lactokinins; tripeptides such as Val-Pro-Pro and Ile-Pro-Pro and the nonapeptide teprotide;
(xix) the ACE inhibitor is one of alacepril, cilazapril, delapril imidapril moexipril, rentiapril, spirapril, temocapril, moveltipril or trandolapril;
(xx) the angiotensin II receptor antagonist is one of candesartan, eprosartan, irbesartan, losartan, olmesartan, telmisartan, or valsartan;
(xxi) the aldosterone antagonist is one of eplerenone, canrenone, or spironolactone;
(xxii) the vasodilator is a cerebral vasodilators such as bencyclane, cinnarizine, citicoline, cyclandelate, ciclonicate, diisopropylamine dichloroacetate, eburnamonine, fasudil, fenoxedil, flunarizine, ibudilast, ifenprodil, lomerizine, nafronyl, nicametate, nicergoline, nimodipine, papaverine, tinofedrine, vincamine, vinpocetine, or viquidil;
(xxiii) the vasodilator is a coronary vasodilators such as amotriphene, bendazol, benfurodil hemisuccinate, benziodarone, chloracizine, chromonar, clobenfural, clonitrate, cloricromen, dilazep, dipyridamole, droprenilamine, efloxate, erythrityl tetranitrate, etafenone, fendiline, floredil, ganglefene, hexestrol bis(beta-diethylaminoethyl) ether, hexobendine, itramin tosylate, khellin, lidoflazine, mannitol hexanitrate, medibazine, nitroglycerin, pentaerythritol tetranitrate, pentrinitrol, perhexiline, pimefylline, prenylamine, propatyl nitrate, trapidil, tricromyl, trimetazidine, trolnitrate phosphate, sildenafil, tadalafil, vardenafil, sodium nitroprusside, isosorbide mononitrate, isosorbide dinitrate, pentaerythritol tetranitrate, theobromine, or visnadine;
(xxiv) the vasodilator is a peripheral vasodilators such as aluminium nicotinate, bamethan, bencyclane, betahistine, bradykinin, brovincamine, bufeniode, buflomedil, butalamine, cetiedil, ciclonicate, cinepazide, cinnarizine, cyclandelate, diisopropylamine dichloroacetate, eledoisin, fenoxedil, flunarizine, hepronicate, ifenprodil, iloprost, inositol niacinate, isoxsuprine, kallidin, kallikrein, moxisylyte, nafronyl, nicametate, nicergoline, nicofuranose, nylidrin, pentifylline, pentoxifylline, piribedil, prostaglandin E1, suloctidil, tolazoline, or xanthinol niacinate; or
(xxV) the renin inhibitor is aliskiren or remikiren.

45. The ICAM-2 binding peptide conjugate of claim 31, wherein the pharmaceutically active moiety is a cancer therapeutic selected from an antimetabolite, an alkaloid, an alkylating agent, an anti-mitotic agent, an antitumor antibiotic, a DNA binding drug, a toxin, an anti-proliferative drug, a DNA antagonist, a radionuclide, a thermoablative agent, a proteolysis targeting chimera (PROTAC), and a nucleic acid inhibitor.

46. The ICAM-2 binding peptide conjugate of claim 45, wherein the alkaloid is selected from the group consisting of duocarmycin, docetaxel, etoposide, irinotecan, paclitaxel, teniposide, topotecan, vinblastine, vincristine, vindesine, and analogs and derivatives thereof.

47. The ICAM-2 binding peptide conjugate of claim 45, wherein the alkylating agent is selected from the group consisting of busulfan, improsulfan, piposulfan, benzodepa, carboquone, meturedepa, uredepa, altretamine, triethylenemelamine, triethylenephosphoramide, triethylenethiophosphorarnide, chlorambucil, chloranaphazine, cyclophosphamide, estramustine, ifosfamide, mechlorethamine, mechlorethamine oxide HCl, melphalan, novemebichin, perfosfamide phenesterine, prednimustine, trofosfamide, uracil mustard, carmustine, chlorozotocin, fotemustine, lomustine, nimustine, semustine ranimustine, dacarbazine, mannomustine, mitobronitol, mitolactol, pipobroman, temozolomide, and analogs and derivatives thereof.

48. The ICAM-2 binding peptide conjugate of claim 45, wherein the antitumor antibiotic is selected from the group consisting of aclacinomycin, actinomycin, anthramycin, azaserine, bleomycin, cactinomycin, calicheamicin, carubicin, carzinophilin, cromomycin, dactinomycin, daunorubicin, 6-diazo-5-oxo-1-norleucine, doxorubicin, epirabicin, idarubicin, menogaril, mitomycin, mycophenolic acid, nogalamycine, olivomycin, peplomycin, pirarubicin, plicamycin, porfiromycin, puromycine, pyrrolobenzodiazepine, streptonigrin, streptozocin, tubercidin, zinostatin, zorubicin, and analogs and derivatives thereof.

49. The ICAM-2 binding peptide conjugate of claim 45, wherein the antimetabolite is selected from the group consisting of from SN-38, denopterin, edatrexate, mercaptopurine (6-MP), methotrexate, piritrexim, pteropterin, pentostatin (2′-DCF), tomudex, trimetrexate, cladridine, fludarabine, thiamiprine, ancitabine, azacitidine, 6-azauridine, carmofur, cytarabine, doxifluridine, emitefur, floxuridine, fluorouracil, gemcitabine, tegafur, hydroxyurea, urethane, and analogs and derivatives thereof.

50. The ICAM-2 binding peptide conjugate of claim 45, wherein the anti-proliferative drug is selected from the group consisting of aceglatone, amsacrine, bisantrene, camptothecin, defosfamide, demecolcine, diaziquone, diflomotecan, eflornithine, elliptinium acetate, etoglucid, etopside, fenretinide, gallium nitrate, hydroxyurea, lamellarin D, lonidamine, miltefosine, mitoguazone, mitoxantrone, mopidamol, nitracrine, pentostatin, phenamet, podophillinic acid 2-ethyl-hydrazide, procarbazine, razoxane, sobuzoxane, spirogermanium, teniposide, tenuazonic acid, triaziquone 2,2′,2″-trichlorotriethylamine, and analogs and derivatives thereof.

51. The ICAM-2 binding peptide conjugate of claim 45, wherein the antimitotic agent is selected from the group consisting of an auristatin, a maytansinoid, a dolastatin, a tubulysin, a taxane, an epothilone, a vinca alkaloid, and analogs and derivatives thereof.

52. The ICAM-2 binding peptide conjugate of any one of claims 31-51, wherein the pharmaceutically active moiety is coupled to or associated with a delivery vehicle.

53. The ICAM-2 binding peptide conjugate of claim 30, wherein the second portion of the conjugate is a delivery vehicle.

54. The ICAM-2 binding peptide conjugate of claim 52 or 53, wherein the delivery vehicle is selected from a nanoparticle, a polymer-based particle, and a lipid-based particle.

55. The ICAM-2 binding peptide conjugate of claim 30, wherein the second portion is a diagnostic moiety.

56. The ICAM-2 binding peptide conjugate of claim 55, wherein the diagnostic moiety is selected from the group consisting of a fluorescent dye, a radioisotope, a contrast agent suitable for imaging, a radionucleotide with chelator, and a photosensitizer.

57. An isolated polynucleotide encoding the ICAM-2 binding peptide of any one of claims 1-29 or the ICAM-2 binding peptide conjugate of claim 30.

58. A vector comprising the isolated polynucleotide of claim 57.

59. A host cell comprising the vector of claim 58.

60. A pharmaceutical composition comprising:
the binding polypeptide of any one of claims 1-29, the ICAM-2 binding peptide conjugate of any one of claims 30-56, the isolated polynucleotide of claim 57, or the vector of claim 58; and
a pharmaceutical carrier.

61. A combination therapeutic comprising:
a binding polypeptide of an any one of claims 1-29 and
a pharmaceutically active agent.

62. A method of inhibiting transplant organ rejection, said method comprising:
administering to a recipient of a donor organ or tissue an effective amount of the ICAM-2 binding peptide conjugate of claim 30, wherein the pharmaceutically active moiety is an immunosuppressant agent, whereby said administering is effective to suppress rejection of the donor organ or tissue.

63. The method according to claim 62, wherein said administering is carried out locally.

64. The method according to claim 62, wherein said administering is carried out systemically.

65. The method according to claim 62, wherein the donor organ is a heart, a kidney, a lung, or a liver (or section thereof).

66. The method according to claim 62, wherein said administering is repeated.

67. A method of treating hypertension, said method comprising:
administering to a patient having hypertension an effective amount of the ICAM-2 binding peptide conjugate of claim 30, wherein the pharmaceutically active moiety is an anti-hypertensive agent, whereby said administering is effective to treat hypertension.

68. The method according to claim 67, wherein said administering is carried out locally.

69. The method according to claim 67, wherein said administering is carried out systemically.

70. A method of treating cancer, said method comprising:
administering to a cancer patient an effective amount of the ICAM-2 binding peptide conjugate of claim 30, wherein the pharmaceutically active moiety is an immunostimulant, anti-angiogenic agent, or chemotherapeutic agent, and said administering is effective to treat the cancer.

71. The method of claim 70, wherein said administering causes delivery of the ICAM-2 binding peptide conjugate to endothelial cells associated with neovascularized primary or secondary tumors.

72. The method of claim 70, wherein the cancer is pancreatic cancer, lung cancer, breast cancer, colon cancer, glioma, solid tumor, melanoma, glioblastoma multiforme, leukemia, renal cell carcinoma, hepatocellular carcinoma, prostate cancer, and myeloma.

73. The method of claim 70, wherein said method further comprising:
administering a cancer therapeutic in conjunction with said ICAM-2 binding peptide conjugate.

74. The method of claim 70, wherein the cancer therapeutic is a chemotherapeutic.

75. A method of treating cancer, said method comprising:
administering to a cancer patient an effective amount of ICAM-2 binding peptide conjugate of claim 30, wherein the conjugate includes a thermo-ablative agent; and
exposing the cancer patient to energy suitable to cause localized heating of the thermo-ablative agent at the site of primary and/or secondary tumors to treat the cancer.

76. The method of claim 75, wherein the cancer is pancreatic cancer, lung cancer, breast cancer, colon cancer, glioma, solid tumor, melanoma, glioblastoma multiforme, renal cell carcinoma, hepatocellular carcinoma, and prostate cancer.
